(19) United States
(12) Patent Application Publication
(10) Pub. No.: US 2008/0058286 A1
(43) Pub. Date: Mar. 6, 2008

(54) Novel applications of omega-3 rich phospholipids

(75) Inventors: Inge Bruheim, Volda (NO); Mikko Grinnari, Espoo (FI)

Correspondence Address: Casimir Jones, S.C., 440 Science Drive, Suite 203, Madison, WI, 53711, US

(73) Assignee: Natural ASA, Lysaker (NO)

(21) Appl. No.: 11/800,369

(22) Filed: May 4, 2007

Related U.S. Application Data
(60) Provisional application No. 60/798,026, filed on May 5, 2006.
(60) Provisional application No. 60/798,027, filed on May 5, 2006.
(60) Provisional application No. 60/798,030, filed on May 5, 2006.
(60) Provisional application No. 60/872,096, filed on Dec. 1, 2006.

(51) Int. Cl. A61K 31/685; A61P 21/00

(52) U.S. Cl. 514/78

(57) ABSTRACT

This invention disclose novel uses of omega-3 rich phospholipids for treating diabetes type II, metabolic syndrome, sustained ventricular tachycardia and inflammatory disease. In addition to improving fertility in asthenozoospermic males. The invention also discloses the use of omega-3 rich phospholipids in healthy subject for improving physical endurances, reducing delayed onset muscle soreness as well as preventing obesity.

This application claims the benefit of U.S. Provisional Applications 60/798,026, 60/798,027, and 60/798,030, all filed May 5, 2006, and U.S. Provisional Application 60/872,096, filed Dec. 1, 2006, each of which is incorporated herein by reference in their entirety.

### FIELD OF THE INVENTION

The invention relates to the use of omega-3 fatty acid compositions, and in particular to phospholipid compositions comprising omega-3 fatty acids, to prevent/treat conditions such as rheumatoid arthritis, diabetes type II, low fertility, cardiac arrhythmia, obesity, blood lipids, insulin resistance, oxidative stress and muscle soreness.

### BACKGROUND OF THE INVENTION

Phospholipids can be isolated from a number of different natural sources such as fish, crustaceans and algae (marine phospholipids). Other sources can be soy, sunflower and maize (vegetable phospholipids). In addition phospholipids can be obtained from eggs. Phospholipids with desired fatty acid residues, so called functional phospholipids, can also be obtained using chemical or enzyme-catalyzed processes [1-5]. The uses of phospholipids enriched with omega-3 fatty acids EPA/DHA have been contemplated and explored for several years both for naturally extracted phospholipids [6-9] and enzymatically synthesized omega-3 rich phospholipids [10]. The uses range from improvement and treatment of cognitive and mental conditions, reduction of inflammation, treatment of inflammatory disease (e.g. rheumatoid arthritis) to treatment of cardiovascular disease and improving quality of life. The driving force behind these developments has been data indicating that phospholipids are superior carrier of fatty acids into tissue such as red blood cells [11] and brain [12] compared to triacylglycerides. The data suggest that marine phospholipids are more bioactive than fish oil, thereby creating a stronger biological effect with same dose. These observations in combination with an increasing number publications showing evidence of positive health effects omega-3 fatty acids in several areas including anti-inflammation [13], cardio-vascular disease [14] and brain function [15] have fueled the research in the area of omega-3 rich functional phospholipids. In other areas, such as treating asthenozoospermic males the results have been mixed and no clear benefit has been shown. The brain, the eye/retina and the testicles are all organs rich in DHA [16]. Likewise in patients with cognitive decline, having reduced levels of DHA and arachidonic acid (ARA) in the brain [17], low fertile males (asthenozoospermic males) show reduced levels of DHA and ARA in their spermatozoa and ejaculate [18]. Fish oil has been tested as a fertility enhancer in humans and animals, however the results obtained have shown some benefits in animals such as improving the motion characteristics of cool stored stallion semen [19]. In humans, the results have not been convincing, showing no effect of pure DHA supplementation on sperm motion characteristics [16]. The reason for lack of observed effect is likely due to the body's inability to incorporate DHA into sperm phospholipids and/or that a DHA should be used in combination with other omega-3 fatty acids such as DHA precursors (EPA). DHA is involved in fertility and is believed to be beneficial during spermatogenesis, influencing membrane fluidity as well as lipid metabolism.

The anti-inflammatory properties of omega-3 fatty acids are well known and the use has been described both for triglycerides and phospholipids. Omega-3 fatty acids have been shown to alleviate the symptoms of a series of autoimmune, atherosclerotic and inflammatory diseases including inflammatory bowel diseases, osteoarthritis and rheumatoid arthritis [20-22]. Suppression of inflammation has been proposed as one of the strategies to slow down the progress of these diseases. Although, much attention has focused on pro-inflammatory pathways that initiate inflammation, relatively little is known about the mechanisms that switch off inflammation and resolve the inflammatory response. The transcription factor NFic B is thought to have a central role in the induction of pro-inflammatory gene expression and has attracted interest as a new target for the treatment of inflammatory disease [23]. When cytokines such as interleukin (IL)-1 and tumor necrosis factor (TNF)-alpha stimulate cells, NF-.κB moves to the nucleus, where it binds to the DNA sequence called the NF-kappaB binding sequence and induces the transcription of the gene, which is believed to regulate the expression of genes such as those for immunoglobulins, inflammatory cytokines (e.g., IL-1 and TNF-α), interferons and cell adhesion factors. Although, the use of marine phospholipids (both extracted and synthesized) for reducing inflammation has been disclosed [8,10], no preferred EPA/DHA ratios has been suggested. New data show that EPA prevents the NF-κB activation [24], therefore, a strong and fast anti-inflammatory agent should have EPA/DHA attached to a phospholipid and have a high EPA/DHA ratio.

Furthermore, numerous studies have demonstrated beneficial effect of omega-3 fatty acids in the area of cardiovascular health e.g. by lowering serum lipids in animal and humans [25]. One of the reasons is due to the fact that omega-3 fatty acids regulate the transcription of genes involved in cholesterol metabolism, fatty acid O-oxidation, and lipogenesis [26]. In addition, modulating the expression of key enzymes such as acetyl CoA, oxidase, acetyl CoA-thioesterases, lipoprotein lipase and carnitine palmitoyl transferases. Furthermore, there are results indicating that omega-3 fatty acids might play a role in weigth management and treating/preventing obesity due to the increased O-oxidation in the mitochondria. DHA rich diets have been found to be suitable in reducing body fat storage, by limiting the accumulation of lipids in adipocytes as well as limiting hyperplasia [27] of the adipocytes.

During the last decades, the use of omega-3 fatty acids have been contemplated in a number of areas, however the results obtained have varied. In the area of omega-3 rich phospholipids, little attention has been paid to the EPA/DHA ratio. New data have been published, showing that this ratio is important in addition it has been shown that phospholipids are superior carriers of fatty acids resulting in higher and faster tissue incorporation. This invention discloses new uses of omega-3, by using omega-3 rich functional phospholipids with optimum EPA/DHA ratios as well as superior results in already disclosed applications using fish oil.

### SUMMARY OF THE INVENTION

An embodiment of the invention is a method to improve the fertility in asthenozoospermic males comprising administering an effective amount of an omega-3 rich phospholipid composition.

Another embodiment of the invention is a method to improve physical endurance/sports performance in a subject comprising administering an effective amount of an omega-3 rich phospholipid composition.

Another embodiment of the invention is a method to alleviate muscle soreness after exercise comprising administering an effective amount of an omega-3 rich phospholipid composition.

Another embodiment of the invention is a method to treat a patient in need of anti-inflammatory/immunosuppressive effects, comprising administering an effective amount of an omega-3 rich phospholipid composition.

Another embodiment of the invention is a method to prevent weight gain/obesity in an individual comprising administering an effective amount of an omega-3 rich phospholipid composition.

Another embodiment of the invention is a method to prevent induction of sustained ventricular tachycardia by administering an omega-3 phospholipid composition.

Another embodiment of the invention is a method to treat metabolic syndrome comprising administering an omega-3 rich phospholipid composition.

Another embodiment of the invention is a method to treat diabetes type II comprising administering an omega-3 rich phospholipid composition.

Accordingly, in some embodiments, the present invention provides a composition comprising phospholipids having the following structure: 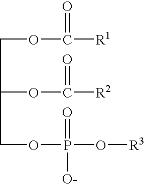
wherein R1 is OH or a fatty acid, R2 is OH or a fatty acid, and R3 is a mixture of H, choline, ethanolamine, inositol and serine, said phospholipid having at least 1% of DHA/EPA at positions R1 and/or R2 and from about 20-50% of OH at positions R1 and/or R2. In some embodiments, the composition further comprises a lipid carrier in a ratio of from 1:10 to 10:1 to said phospholipids. In some embodiments, the lipid carrier and said phospholipids are in a ratio of from about 5:1 to 1:5. In some embodiments, the composition comprises from about 20% to about 90% of said phospholipid composition and from about 10% to about 50% of said lipid carrier. The present invention is not limited to any particular lipid carrier. In some embodiments, the lipid carrier is selected from the group consisting of a triglyceride, a diglyceride, an ethyl ester, and a methyl ester and combinations thereof. In some embodiments, the composition provides higher uptake of omega-3 fatty acids into plasma as compared to natural marine phospholipids when administered to subjects. In some embodiments, the composition improves the AA/EPA ratio in plasma phospholipids when administered to subjects as compared to natural marine phospholipids. In some embodiments, the composition increases the concentration of omega-3 fatty acids in tissues when administered to subjects as compared to natural marine phospholipids. In some embodiments, the composition reduces the concentration of biomarkers of inflammation when administered to subjects as compared to natural marine phospholipids. In some embodiments, the present invention provides a food product comprising the foregoing compositions. In some embodiments, the present invention provides an animal feed comprising the foregoing compositions. In some embodiments, the present invention provides a food supplement comprising the foregoing compositions. In some embodiments, the present invention provides a pharmaceutical composition comprising the foregoing compositions.

In some embodiments, the present invention provides methods of preparing a bioavailable omega-3 fatty acid composition comprising: a) providing a purified phospholipid composition comprising omega-3 fatty acid residues and a purified triglyceride composition comprising omega-3 fatty acid residues; b) combining said phospholipid composition and said triglyceride composition to form a bioavailable omega-3 fatty acid composition. In some embodiments, the bioavailable phospholipid composition is one of the compositions described above. In some embodiments, the methods further comprise the step of encapsulating said bioavailable omega-3 fatty acid composition. In some embodiments, the bioavailable omega-3 fatty acid composition has increased bioavailability as compared to purified triglycerides or phospholipids comprising omega-3 fatty acid residues. In some embodiments, the methods further comprise the step of packaging the bioavailable omega-3 fatty acid composition for use in functional foods. In some embodiments, the methods further comprise the step of assaying the bioavailable omega-3 fatty acid composition for bioavailability. In some embodiments, the methods further comprise administering the bioavailable omega-3 fatty acid composition to a patient. In some embodiments, the present invention provides a food product, animal feed, food supplement or pharmaceutical composition made by the foregoing process.

In some embodiments, the present invention provides methods for reducing symptoms of cognitive dysfunction in a child comprising administering an effective amount of a marine phospholipid composition, wherein said symptoms are selected from the group consisting of ability to complete task, ability to stay on task, ability to follow instructions, ability to complete assignments, psychomotor function, long term memory, short term memory, ability to make a decision, ability to follow through on decision, ability to self-sustain attention, ability to engage in conversations, sensitivity to surroundings, ability to plan, ability to carry out plan, ability to listen, interruptions in social situations, temper tantrums, level/frequency of frustration, level/frequency restlessness, frequency/level fidgeting, ability to exhibit delayed gratification, aggressiveness, demanding behavior/frequency of demanding behavior, sleep patterns, restive sleep, interrupted sleep, awakening behavior, disruptive behavior, ability to exhibit control in social situations, ability to extrapolate information and ability to integrate information. In some embodiments, the child exhibits one or more symptoms of Attention Deficit Hyperactivity Disorder (ADHD), is suspected of having ADHD, or has been diagnosed with ADHD. In some embodiments, the child exhibits one or more symptoms of autistic spectrum disorder, is suspected of having autistic spectrum disorder, or has been diagnosed with autistic spectrum disorder. In further embodiments, the present invention provides methods of increasing cognitive performance in an aging mammal comprising administering an effective amount of a marine phospholipid composition. In some embodiments, the cognitive performance is selected from the group consisting of memory loss, forgetfulness, short-term memory loss, aphasia, disorientation, disinhibition, and behavioral changes. In some embodiments, the mammal is a human. In some embodiments, the mammal is a pet selected from the group consisting of cats and dogs. In some embodiments, the mammal has symptoms of age-associated memory impairment or decline.

The foregoing methods are not limited to the use of any particular marine phospholipid composition. In some embodiments, the marine phospholipid composition comprises phospholipids having the following structure: 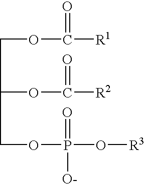
wherein R1 is OH or a fatty acid, R2 is OH or a fatty acid, and R3 is a mixture of H, choline, ethanolamine, inositol and serine, said phospholipid having at least 1% of omega-3 fatty acid moieties at positions R1 and/or R2. In some embodiments, the phospholipid composition comprises from about 20-50% of OH at positions R1 and/or R2. In some embodiments, the phospholipid composition further comprises a lipid carrier. In some embodiments, the phospholipid composition is prepared from natural marine phospholipids isolated from a marine organism. In some embodiments, the phospholipid composition is enzymatically prepared by reacting lecithin with DHA and EPA in the presence of an enzyme. In some embodiments, the lecithin is soybean or egg lecithin. In some embodiments, the omega-3 fatty acid moieties are selected from the group of EPA and DHA and combination thereof. In some embodiments, the effective amount of said phospholipid composition comprises from about 300 to about 1000 mg omega-3 fatty acids. In some embodiments, the phospholipid composition is administered orally. In some embodiments, the phospholipid composition is provided in a gel capsule or pill.

In some embodiments, the present invention provides methods of treating a subject by administration of a marine phospholipid composition comprising administering a marine phospholipid composition to said subject under conditions such that a desired condition is improved, wherein said conditions is selected from the group consisting of fertility, physical endurance, sports performance, muscle soreness, inflammation, auto-immune stimulation, metabolic syndrome, obesity and type II diabetes. In some embodiments, the subject is a human. In some embodiments, the subject is a companion animal. The present invention is not limited to any particular marine phospholipid composition. In some embodiments, the marine phospholipid composition comprises phospholipids having the following structure: 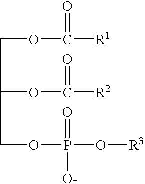
wherein R1 is OH or a fatty acid, R2 is OH or a fatty acid, and R3 is a mixture of H, choline, ethanolamine, inositol and serine, said phospholipid having at least 1% of omega-3 fatty acid moieties at positions R1 and/or R2. In some embodiments, the phospholipid composition comprises from about 20-50% of OH at positions R1 and/or R2. In some embodiments, the phospholipid composition is prepared from natural marine phospholipids isolated from a marine organism. In some embodiments, the composition further comprises a lipid carrier. In some embodiments, the phospholipid composition is enzymatically prepared by reacting lecithin with DHA and EPA in the presence of an enzyme. In some embodiments, the lecithin is soybean or egg lecithin. In some embodiments, the omega-3 fatty acid moieties are selected from the group of EPA and DRA and combination thereof. In some embodiments, the effective amount of said phospholipid composition comprises from about 300 to about 1000 mg omega-3 fatty acids. In some embodiments, the phospholipid composition is administered orally. In some embodiments, the phospholipid composition is provided in a gel capsule or pill. In some embodiments, the human is a male.

In some embodiments, the present invention provides methods for prophylactically treating a subject by administration of a marine phospholipid composition comprising administering a marine phospholipid composition to a subject under conditions such that an undesirable condition is prevented, wherein said undesirable condition is selected from the group consisting of weight gain, infertility, obesity, metabolic syndrome, diabetes type II, mortality in subjects with a high risk of sudden cardiac death, and induction of sustained ventricular tachycardia. In some embodiments, the subject is at risk for developing a condition selected from the group consisting of weight gain, obesity, metabolic syndrome, diabetes type II, mortality in subjects with a high risk of sudden cardiac death, and induction of sustained ventricular tachycardia.

In some embodiments, the subject is a human. In some embodiments, the subject is a companion animal. The present invention is not limited to any particular marine phospholipid composition. In some embodiments, the marine phospholipid composition comprises phospholipids having the following structure: 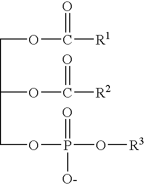
wherein R1 is OH or a fatty acid, R2 is OH or a fatty acid, and R3 is a mixture of H, choline, ethanolamine, inositol and serine, said phospholipid having at least 1% of omega-3 fatty acid moieties at positions R1 and/or R2. In some embodiments, the phospholipid composition comprises from about 20-50% of OH at positions R1 and/or R2. In some embodiments, the phospholipid composition is prepared from natural marine phospholipids isolated from a marine organism. In some embodiments, the composition further comprises a lipid carrier. In some embodiments, the phospholipid composition is enzymatically prepared by reacting lecithin with DHA and EPA in the presence of an enzyme. In some embodiments, the lecithin is soybean or egg lecithin. In some embodiments, the omega-3 fatty acid moieties are selected from the group of EPA and DHA and combination thereof. In some embodiments, the effective amount of said phospholipid composition comprises from about 300 to about 1000 mg omega-3 fatty acids. In some embodiments, the phospholipid composition is administered orally. In some embodiments, the phospholipid composition is provided in a gel capsule or pill.

### DEFINITIONS

As used herein, “phospholipid” refers to an organic compound having the following general structure: 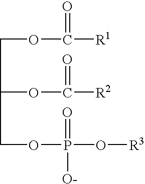
wherein R1 is a fatty acid residue, R2 is a fatty acid residue or —OH, and R3 is a —H or nitrogen containing compound choline (HOCH₂CH₂N⁺(CH₃)₃OH⁻), ethanolamine (HOCH₂CH₂NH₂), inositol or serine. R1 and R2 cannot simultaneously be OH. When R3 is an —OH, the compound is a diacylglycerophosphate, while when R3 is a nitrogen-containing compound, the compound is a phosphatide such as lecithin, cephalin, phosphatidyl serine or plasmalogen.

The R1 site is herein referred to as position 1 of the phospholipid, the R2 site is herein referred to as position 2 of the phospholipid, and the R3 site is herein referred to as position 3 of the phospholipid.

As used herein, the term omega-3 fatty acid refers to polyunsaturated fatty acids that have the final double bond in the hydrocarbon chain between the third and fourth carbon atoms from the methyl end of the molecule. Non-limiting examples of omega-3 fatty acids include, 5,8,11,14,17-eicosapentaenoic acid (EPA), 4,7,10,13,16,19-docosahexanoic acid (DHA) and 7,10,13,16,19-docosapentanoic acid (DPA).

As used herein, the term “functional food” refers to a food product to which a biologically active supplement has been added.

As used herein, the term “fish oil” refers to any oil obtained from a marine source e.g. tuna oil, seal oil and algae oil.

As used herein, the term “lipase” refers to any enzyme capable of hydrolyzing fatty acid esters

As used herein, the term “food supplement” refers to a food product formulated as a dietary or nutritional supplement to be used as part of a diet.

As used herein, the term “extracted marine phospholipid” refers to a composition characterized by being obtained from a natural source such as krill, fish meal, pig brain or eggs.

As used herein, the term “acylation” means fatty acids attached to the phospholipid. 100% acylation means that there are no lyso- or glycero-phospholipids.

As used herein, the term “metabolic syndrome” refers a to syndrome marked by the presence of usually three or more of a group of factors (as high blood pressure, abdominal obesity, high triglyceride levels, low HDL levels, and high fasting levels of blood sugar) that are linked to an increased risk of cardiovascular disease and type 2 diabetes.

### DESCRIPTION OF THE INVENTION

An embodiment of the invention is the use of omega-3 rich phospholipids to improve fertility in healthy and asthenozoospermic humans and animals. Testicular long chain PUFAs are of special interest because there is a high rate of production of prostaglandins from the omega-6 PUFA (arachidonic acid mainly) into the semen or seminal fluid. High rate of prostaglandin production does not indicate an active inflammatory process but a stimulus for the uterus smooth muscle to favour male fertility [28]. In addition, arachidonic acid, prostaglandins and leukotrienes have been implicated in mediating the stimulatory actions of luteinizing hormone on testicular steroid synthesis. An omega-3 induced decrease of arachidonic acid as observed in other tissue could be detrimental to the male fertility, if it occurred also in testis. Furthermore, testicular tissue has also a high level of DPA (22:5 omega-6), which may serve as a reservoir for arachidonic acid. Arachidonic acid could be formed according to the need, through the retroconversion mechanism in the peroxisomes. A similar mechanism may take place with DHA to form EPA in other tissues. Data disclosed in this application (table 4) show an increase of EPA and DHA and a small decrease of arachidonic acid in the total lipids fraction when omega-3 fatty acids are fed. However, there is no change in arachidonic acid levels in the phospholipids (PL) when TG-oil are fed and interestingly a significant increase in the PL-EPA (EPA rich phospholipids) and PL-DHA (DHA rich phospholipids) group (table 5). This can also be seen in the sn-2 positional analysis on the phospholipids (table 6) which is very important as prostaglandins are produced from ARA, which are catalytically hydrolyzed from position 2 on the phospholipid by the action of phospholipase A2. The phospholipase A2 is released after stimuli at the cell wall, it then moves to the nuclear membrane where the hydrolysis of the phospholipid takes place. Furthermore, DPA n-6 concentration in total lipids was not influenced by omega-3 supplementation but there was a significant increase in DPA in the PL-EPA group. Overall, these data show that the diets with omega-3 phospholipids changed the arachidonic and DPA n-6 concentrations in a way that would predict positive effects on male fertility.

In another embodiment, this invention provides methods to reduce inflammation/treat an inflammatory disorder in an animal or a human subject by administering an omega-3 rich phospholipid characterized by having a high EPA/DHA ratio, preferably at least 2:1. This invention discloses that after administering marine phospholipids with a EPA/DHA ratio of 2:1 for 1 week a number of genes involved in the inflammatory response are regulated in a positive way. Furthermore, it is disclosed that marine phospholipids with EPA/DHA ratio of 1:1 do not regulate any genes involved in the inflammatory response. Examples of the proteins regulated by the high EPA phospholipid are the CCAAT/enhancer binding protein (C/EBP), monoglyceride lipase (Mgll), Nuclear Factor-kappaB activating protein (NF-κB AP-1) and Tnf receptor-associated factor 6 (Traf6). C/EBP plays a key role in acute-phase response to inflammatory cytokine IL-6 [29], Traf6 positively regulates the biosynthesis of interleukin-6 and interleukin-12, as well as the 1-kappaB kinase/NF-kappaB cascade [30] and NF-κB AP-1 induce the expression of genes involved in inflammation [31].

Another embodiment of the invention is the use of marine phospholipids to alleviate muscle soreness/muscle pain after physical exercise/sports activity. Delayed onset muscle soreness normally increases in intensity during the first 24 hours after exercise and peaks before 72 hours [32], and then subsides so that by 5-7 days post exercise it is gone. The discomfort ranges from mild to extreme soreness, which prevents the use of the muscle. The reason for the soreness is damage to the connective and/or contractile tissues and that initiates inflammation [33]. Injury in a muscle causes monocytes to migrate to the area and secrets large amounts of pro-inflammatory prostaglandins. This invention discloses that administration of omega-3 rich phospholipids with EPA:DHA ratio of at least 2:1 for 1 week reduces the expression of enzymes in the inflammatory response such as the expression proteins in the NF-κB pathway. Furthermore, it has been shown that the concentration of DHA in red blood cells after a bolus intake of DHA-PC peaked after 9 hours, whereas similar intake of DHA in the form of triglycerides peaked after 12 hours [1,1]. Hence, omega-3 rich phospholipids, preferably with an EPA:DHA ratio of 2:1, are suitable for a rapid reduction in inflammation, preferably in the area of reducing pain, more preferably in the area of reducing delayed onset muscles soreness after physical exercise.

Another embodiment of the invention is to treat conditions involving inflamed joints such as rheumatoid arthritis and osteoarthritis.

Another embodiment of the invention is the use of omega-3 phospholipids to improve physical performance/endurance e.g. in athletes. Studies have shown that incorporation of omega-3 fatty acids into the membrane of RBCs increase the deformability of RBCs [34] which again facilitates the transport of RBCs through the capillary bed [35]. This effect enhances the oxygen delivery to contracting muscle which may have a benefit on improving physical performance. This invention discloses that mice fed a diet comprising omega-3 rich phospholipids perform better than mice fed omega-3 rich triglycerides i.e. increased submaximal endurance in a treadmill running test.

Another embodiment of the invention is the use of marine phospholipids to prevent obesity and for weight management in humans and animals. This invention discloses that omega-3 rich phospholipids (EPA:DHA ratio of 2:1) regulate several genes linked to lipid metabolism in a positive way such as gamma-butyrobetaine hydroxylase and guanine nucleotide binding protein. The results show that guanine nucleotide binding protein is down regulated. This results in an increased inhibition of adenylate cyclase (AC). AC catalyzes the conversion of ATP to 3′,5′-cyclic AMP (cAMP) and pyrophosphate. cAMP is an important molecule in eukaryotic signal transduction and is responsible for the intracellular mediation of hormonal effects on various cellular processes such as lipid metabolism, membrane transport, and cell proliferation [36]. Furthermore, the level of gamma-butyrobetaine hydroxylase is increased leading to increased biosynthesis of L-carnitine (3-hydroxy-4-N-trimethylaminobutyrate) [37]. Increased carnitine levels result in increased β-oxidation [38], since carnitine is responsible for transport of fatty acids into a cell's mitochondria. This invention disclose that marine phospholipids can increase β-oxidation of fatty acids in mitochondria which may represents shift in fuel use from glucose and amino acids to fats. Marine phospholipids can therefore be used to prevent weight gain or obesity in combination with a high fat diet.

In yet another embodiment, marine phospholipids are provided as a prophylactic treatment of rapid heart beat (sustained ventricular tachycardia) in patients at high risk of sudden cardiac death. Published data have shown that omega-3 fatty acids reduce cardiovascular mortality [39], and that incidences of ventricular tachycardia can be reduced in patients after infusion of omega-3 fatty acids [40]. Due to the rapid incorporation of marine phospholipids into RBCs [11], phospholipids are more suitable than triglycerides when a rapid/acute/immediate effect is needed. Patients with sustained ventricular tachycarida in patients with a high risk of sudden cardiac death, hence it is likely that marine phospholipids will be more efficient preventing death than fish oil. This invention discloses that heart total lipids and phospholipids (table 7 and table 8, respectively) showed a strong increase of EPA and DHA with a concomitant decrease of arachidonic acid when omega-3 supplements were fed. The strong decrease in the omega-6/omega-3 ratio in heart lipids is important considering the possible impact on the anti-inflammatory potential. Observed change in heart tissue fatty acids (increase of fatty acids with 6 or 5 double bonds) also suggests a possible increase in membrane fluidity. This change was most striking in the PL-DHA group where the increase of DHA was significantly higher than the increase in the TG-oil and PL-EPA groups. The fluidity of myocardium cell membrane seems to play an important role in controlling arrhythmia. Ventricular arrhythmia, is one of the main causes of sudden cardiac death. Furthermore, atrial fibrillation is another pathological state with a high incidence and important health consequences.

Another embodiment of the invention is the use of marine phospholipids to reduce the symptoms of metabolic syndrome and/or diabetes type II. Metabolic syndrome is considered as a combination of metabolic disorders that increases a subject's risk for cardiovascular disease and type 2 diabetes. The criteria for metabolic syndrome are fasting hyperglycemia, high blood pressure, central obesity, decreased HDL cholesterol, increased triglycerides and elevated uric acid levels. Hence, Zücker diabetic fatty rat can be used to monitor the effect of dietary intervention on the development and progress of metabolic syndrome. This invention discloses that omega-3 phospholipids are superior to omega-3 triglycerides in alleviating insulin resistance, improving cholesterol profile and reducing plasma triglycerides.

In some embodiments, the marine phospholipid compositions are derived from marine organisms such as fish, fish eggs, shrimp, krill, etc. In some embodiments, the marine phospholipids comprise a mixture of phosphatidylserine (PS), phosphatidylcholine (PC), phosphatidyl inositol (PI), and phosphatidylethanolamine (PE). Indeed, the present invention presents the surprising results that phospholipid compositions comprising a mixture of PC, PS, PI and PE are bioavailable and bioefficient. This results in an important advantage over phospholipid compositions synthesized or containing, for example, pure PS, PC, or PI which can be expensive and difficult to make. In some embodiments, the methods of the present invention utilize novel marine lipid compositions comprising an omega-3 containing phospholipid and a triacylglyceride (TG) in a ratio from about 1:10 to 10:1. Preferably the ratio is in the range of from about 3:1 to 1:3, more preferably the ratio is in the range of about 1:2 to 2:1. Preferably, the TG is a fish oil such as tuna oil, herring oil, menhaden oil, krill oil, cod liver oil or algae oil. However, this invention is not limited to omega-3 containing oils as other TG sources are contemplated such as vegetable oils. In some embodiments, the phospholipids in the composition have the following structure: 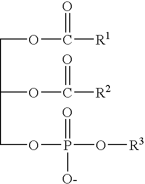
wherein R1 is OH or a fatty acid, R2 is OH or a fatty acid, and R3 is a mixture of H, choline, ethanolamine, inositol and serine. Attached to position 1 or position 2 are least 1% omega-3 fatty acids, preferably at least 5%, more preferably at least 10% omega-3 fatty acids, up to about 15%, 20%, 30%, 40%, 50%, or 60% omega-3 fatty acids. The omega-3 fatty acids can be EPA, DHA, DPA or C18:3 (n-3), most preferably the omega-3 fatty acids are EPA and DHA. The phospholipid composition preferably contains OH in position 1 or position 2 in a range of preferably about 15% to 60%, and more preferably from about 20% to 50% in order to maximize absorption in-vivo.

Transesterification of phosphatidylcholine (PC) under solvent free conditions has been performed by Haraldsson et al in 1999 [15], with the results of high incorporation of EPA/DHA and with the following hydrolysis profile PC/LPC/GPC=39/44/17. Extensive hydrolysis and by-product formation is generally considered a problem with transesterification reactions, resulting in low product yields. This invention discloses a process for transesterification of crude soybean lecithin (mixture of PC, PE and PI). In the first step, the lecithin is hydrolyzed using a lipase in the presence of water (pH=8). The use of a variety of lipases is contemplated, including, but not limited to, Thermomyces Lanuginosus lipase, Rhizomucor miehei lipase, Candida Antarctica lipase, Pseudomonas fluorescence lipase, and Mucor javanicus lipase. The first step takes around 24 hours and results in a product comprising predominantly of lyso-phospholipids and glycerophospholipids such as PC/LPC/GPC=0/15/85. In the second step, free fatty acids are added such as EPA and DHA, however any omega-3 fatty acid is contemplated. Next a strong vacuum is applied to the reaction vessel for 72 hours. However, the reaction length can be varied in order to obtain a composition with the desired amount of phospholipids and lyso-phospholipids. By extending the reaction time beyond 72 hours, a product comprising more than 65% phospholipids can be obtained. Next, a lipid carrier is added to the reaction mixture in order to reduce the viscosity of the solution. The added amount of triglycerides can be 10%, 20%, 30%, 40% or more, it depends on the requested viscosity of the final product. The lipid carrier can be a fish oil such as tuna oil, menhaden oil and herring oil, or any triglyceride, diglyceride, ethyl- or methylester of a fatty acid. In the final step, the product is subjected to a molecular distillation and the free fatty acids are removed, resulting in a final product comprising of phospholipids (lyso-phospholipids and phospholipids) and triglycerides in a ratio of preferably 2:1.

In some embodiments, the compositions of this invention are contained in acceptable excipients and/or carriers for oral consumption. The actual form of the carrier, and thus, the compositions itself, is not critical. The carrier may be a liquid, gel, gelcap, capsule, powder, solid tablet (coated or non-coated), tea, or the like. The composition is preferably in the form of a tablet or capsule and most preferably in the form of a hard gelatin capsule. Suitable excipient and/or carriers include maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, lactose, methylcellulose, povidone, carboxymethylcellulose, corn starch, and the like (including mixtures thereof). Preferred carriers include calcium carbonate, magnesium stearate, maltodextrin, and mixtures thereof. The various ingredients and the excipient and/or carrier are mixed and formed into the desired form using conventional techniques. The tablet or capsule of the present invention may be coated with an enteric coating that dissolves at a pH of about 6.0 to 7.0. A suitable enteric coating that dissolves in the small intestine but not in the stomach is cellulose acetate phthalate. Further details on techniques for formulation for and administration may be found in the latest edition of Remington's Pharmaceutical Sciences (Maack Publishing Co., Easton, Pa.).

In other embodiments, the supplement is provided as a powder or liquid suitable for adding by the consumer to a food or beverage. For example, in some embodiments, the dietary supplement can be administered to an individual in the form of a powder, for instance to be used by mixing into a beverage, or by stirring into a semi-solid food such as a pudding, topping, sauce, puree, cooked cereal, or salad dressing, for instance, or by otherwise adding to a food.

The compositions of the present invention may also be formulated with a number of other compounds. These compounds and substances add to the palatability or sensory perception of the particles (e.g., flavorings and colorings) or improve the nutritional value of the particles (e.g., minerals, vitamins, phytonutrients, antioxidants, etc.).

### EXPERIMENTAL

### Example 1

Marine phospholipids were prepared using either 40% soy PC (American Lecithin Company Inc, Oxford, Conn., USA) (MPL1) or 96% pure soy PC (Phospholipid GmbH, Koln, Germany) (MPL2) according to a method described by others [4]. Fatty acid content and the level of bi-products are shown in table 1. The MPL treatments consisted of a mixture of phospholipids, lyso-phospholipids and glycerol-phospholipids. Looking only at the PC/LPC/GPC relationship, it was 64/33/2 and 42/40/18 for MPL1 and MPL2, respectively. Finally, all three treatments were emulsified into skimmed milk.

TABLE 1
Composition of the phospholipids used in example 1

| | | | 18:3 | | |
|---|---|---|---|---|---|
| Composition | PC/LPC/GPC | 18:2 (n-6) | (n-3) | EPA | DHA |
| MPL1 | 64/33/2 | 129 mg/g | 9 mg/g | 51 mg/g | 171 mg/g |
| MPL2 | 42/40/18 | 124 mg/g | 9 mg/g | 96 mg/g | 96 mg/g |

18 newly weaned Sprague-Dawley rats were fed the milk emulsions for 1 week. Each rat was placed in its own cage to ensure that they got an even amount of test substance and the milk was consumed by the rat pups ad libitum. After 1 week, the experiment was terminated and the rats were decapitated. The animals were kept without food for 24 hours before sampling. Entire livers were collected and frozen immediately using liquid nitrogen (stored at −65° C.). Total RNA was isolated from the liver samples according to the Quiagen Rnaesy Midi Kit Protocol. The RNA samples were quantified and quality measured by Nanopropand Bioanalyzer. The isolated RNA was hybridized onto a gene chip RAE230 2.0 from Affymetrix (Santa Clara, Calif., USA). The expression level of each gene was measured using an Affymetrix GeneChip 3000 7G scanner. The results were suitable for all chips except 2 and they were excluded from the trial. Using statistical tools a list of genes expressed differentially between omega-3 rich phospholipids versus control was obtained. The results are based on (log) probe set summary expression measures, computed by RMA, and linear models are fitted using Empirical Bayes methods for borrowing strength across genes (using the Limma package in R). The p-value are adjusted for multiple testing using the Benjamini-Hochberg-method, controlling the False Discovery Rate (FDR), where FDR=the proportion of null-hypotheses of no DE that are falsely rejected. MPL2 regulated 401 genes versus the control (table 2). A number of genes listed are involved maintenance of the cell, in transcription and protein synthesis as well as signaling pathways. Others are involved in regulation of metabolism and the inflammatory response such as Tnf receptor-associated factor 6 (Traf6_predicted) (fold change of 0.53), guanine nucleotide binding protein alpha inhibiting 2 (Gnai2) (fold change of 0.6, gamma-butyrobetaine hydroxylase (Bbox1) (fold change of 1.32), monoglyceride lipase (Mgll) (fold change 0.52), nuclear NF-kappaB activating protein (fold change 0.65) and CCAAT/enhancer binding protein (C/EBP) (fold change of 0.66).

TABLE 2
List of genes differentially expressed (DE) by MPL2 versus control.

| | | | | | | Fold | Affy | |
|---|---|---|---|---|---|---|---|---|
| Gene-ID | Gene name | t | p-value | FDR | SLR | change | Fold.change | df |
| 1367588_a_at | ribosomal protein L13A | −5.22 | 0.00005 | 0.00568 | −0.44 | 0.74 | −1.36 | 14 |
| 1367844_at | guanine nucleotide binding | −5.47 | 0.00003 | 0.00417 | −0.54 | 0.69 | −1.46 | 14 |
| | protein, alpha inhibiting 2 | | | | | | | |
| 1367958_at | abl-interactor 1 | −6.42 | 0.00000 | 0.00119 | −0.69 | 0.62 | −1.62 | 14 |
| 1367971_at | protein tyrosine phosphatase | −6.01 | 0.00001 | 0.00190 | −0.36 | 0.78 | −1.28 | 14 |
| | 4a2 | | | | | | | |
| 1368057_at | ATP-binding cassette, sub- | −5.06 | 0.00007 | 0.00688 | −0.54 | 0.69 | −1.45 | 14 |
| | family D (ALD), member 3 | | | | | | | |
| 1368405_at | v-ral simian leukemia viral | −4.86 | 0.00011 | 0.00913 | −0.44 | 0.74 | −1.36 | 14 |
| | oncogene homolog A (ras | | | | | | | |
| | related) | | | | | | | |
| 1368646_at | mitogen-activated protein | 4.98 | 0.00008 | 0.00772 | 0.70 | 1.62 | 1.62 | 14 |
| | kinase 9 | | | | | | | |
| 1368649_at | dyskeratosis congenita 1, | −6.73 | 0.00000 | 0.00080 | −0.53 | 0.69 | −1.44 | 14 |
| | dyskerin | | | | | | | |
| 1368662_at | ring finger protein 39 | −7.01 | 0.00000 | 0.00053 | −0.61 | 0.66 | −1.52 | 14 |
| 1368703_at | enigma homolog | −5.38 | 0.00003 | 0.00450 | −0.76 | 0.59 | −1.70 | 14 |
| 1368824_at | caldesmon 1 | −7.18 | 0.00000 | 0.00043 | −1.00 | 0.50 | −2.00 | 14 |
| 1368841_at | transcription factor 4 | −4.94 | 0.00009 | 0.00828 | −0.38 | 0.77 | −1.31 | 14 |
| 1368867_at | GERp95 | −7.83 | 0.00000 | 0.00019 | −0.85 | 0.56 | −1.80 | 14 |
| 1369094_a_at | protein tyrosine phosphatase, | −7.22 | 0.00000 | 0.00042 | −0.97 | 0.51 | −1.96 | 14 |
| | receptor type, D | | | | | | | |
| 1369127_a_at | prostaglandin F receptor | 4.85 | 0.00011 | 0.00921 | 0.45 | 1.37 | 1.37 | 14 |
| 1369174_at | SMAD, mothers against DPP | −5.19 | 0.00005 | 0.00581 | −0.38 | 0.77 | −1.30 | 14 |
| | homolog 1 (Drosophila) | | | | | | | |
| 1369227_at | Choroidermia | 5.04 | 0.00007 | 0.00718 | 0.47 | 1.39 | 1.39 | 14 |
| 1369249_at | progressive ankylosis homolog | 5.36 | 0.00004 | 0.00467 | 0.48 | 1.39 | 1.39 | 14 |
| | (mouse) | | | | | | | |
| 1369501_at | zinc finger protein 260 | 5.17 | 0.00005 | 0.00595 | 0.41 | 1.33 | 1.33 | 14 |
| 1369517_at | pleckstrin homology, Sec7 and | 4.93 | 0.00009 | 0.00829 | 0.48 | 1.40 | 1.40 | 14 |
| | coiled/coil domains 1 | | | | | | | |
| 1369546_at | butyrobetaine (gamma), 2- | 4.96 | 0.00009 | 0.00811 | 0.40 | 1.32 | 1.32 | 14 |
| | oxoglutarate dioxygenase 1 | | | | | | | |
| | (gamma-butyrobetaine | | | | | | | |
| | hydroxylase) | | | | | | | |
| 1369628_at | synaptic vesicle glycoprotein | −7.20 | 0.00000 | 0.00042 | −1.11 | 0.46 | −2.15 | 14 |
| | 2b | | | | | | | |
| 1369689_at | N-ethylmaleimide sensitive | 6.22 | 0.00001 | 0.00155 | 0.66 | 1.58 | 1.58 | 14 |
| | fusion protein | | | | | | | |
| 1369736_at | epithelial membrane protein 1 | 5.74 | 0.00002 | 0.00286 | 0.62 | 1.54 | 1.54 | 14 |
| 1369775_at | nuclear ubiquitous casein | −7.56 | 0.00000 | 0.00027 | −0.79 | 0.58 | −1.73 | 14 |
| | kinase and cyclin-dependent | | | | | | | |
| | kinase substrate | | | | | | | |
| 1370184_at | cofilin 1 | −6.07 | 0.00001 | 0.00178 | −0.38 | 0.77 | −1.30 | 14 |
| 1370260_at | adducin 3 (gamma) | −5.50 | 0.00003 | 0.00399 | −0.76 | 0.59 | −1.70 | 14 |
| 1370328_at | Dickkopf homolog 3 (Xenopus | 4.80 | 0.00012 | 0.00964 | 0.59 | 1.51 | 1.51 | 14 |
| | laevis) | | | | | | | |
| 1370717_at | AP1 gamma subunit binding | 6.00 | 0.00001 | 0.00192 | 0.58 | 1.50 | 1.50 | 14 |
| | protein 1 | | | | | | | |
| 1370831_at | monoglyceride lipase | −5.47 | 0.00003 | 0.00414 | −0.94 | 0.52 | −1.92 | 14 |
| 1370901_at | similar to hypothetical protein | −4.83 | 0.00012 | 0.00948 | −0.34 | 0.79 | −1.27 | 14 |
| | MGC36831 (predicted) | | | | | | | |
| 1370946_at | nuclear factor I/X | −10.64 | 0.00000 | 0.00002 | −1.17 | 0.45 | −2.25 | 14 |
| 1370949_at | myristoylated alanine rich | −7.58 | 0.00000 | 0.00026 | −1.17 | 0.44 | −2.26 | 14 |
| | protein kinase C substrate | | | | | | | |
| 1370993_at | laminin, gamma 1 | 6.13 | 0.00001 | 0.00171 | 0.63 | 1.54 | 1.54 | 14 |
| 1371034_at | one cut domain, family | −5.65 | 0.00002 | 0.00327 | −1.77 | 0.29 | −3.40 | 14 |
| | member 1 | | | | | | | |
| 1371059_at | protein kinase, cAMP- | 5.24 | 0.00005 | 0.00556 | 0.48 | 1.40 | 1.40 | 14 |
| | dependent, regulatory, type 2, | | | | | | | |
| | alpha | | | | | | | |
| 1371345_at | methyl-CpG binding domain | −5.32 | 0.00004 | 0.00491 | −0.34 | 0.79 | −1.27 | 14 |
| | protein 3 (predicted) | | | | | | | |
| 1371361_at | similar to tensin | −7.21 | 0.00000 | 0.00042 | −0.60 | 0.66 | −1.51 | 14 |
| 1371394_x_at | similar to Ab2-143 | −5.11 | 0.00006 | 0.00645 | −0.63 | 0.64 | −1.55 | 14 |
| 1371397_at | nitric oxide synthase | −5.53 | 0.00002 | 0.00383 | −0.34 | 0.79 | −1.26 | 14 |
| | interacting protein (predicted) | | | | | | | |
| 1371428_at | | −5.76 | 0.00001 | 0.00276 | −0.37 | 0.77 | −1.29 | 14 |
| 1371430_at | dystroglycan 1 | −5.46 | 0.00003 | 0.00417 | −0.62 | 0.65 | −1.53 | 14 |
| 1371432_at | | −4.95 | 0.00009 | 0.00811 | −0.36 | 0.78 | −1.28 | 14 |
| 1371452_at | bone marrow stromal cell- | −5.05 | 0.00007 | 0.00705 | −0.46 | 0.73 | −1.37 | 14 |
| | derived ubiquitin-like protein | | | | | | | |
| 1371573_at | ribosomal protein L36a | −5.90 | 0.00001 | 0.00221 | −0.40 | 0.76 | −1.32 | 14 |
| | (predicted) | | | | | | | |
| 1371589_at | Ubiquitin-Like 5 Protein | −5.28 | 0.00004 | 0.00518 | −0.57 | 0.68 | −1.48 | 14 |
| 1371590_s_at | Ubiquitin-Like 5 Protein | −4.94 | 0.00009 | 0.00829 | −0.39 | 0.76 | −1.31 | 14 |
| 1371779_at | sorting nexin 6 (predicted) | 5.64 | 0.00002 | 0.00329 | 0.63 | 1.55 | 1.55 | 14 |
| 1371826_at | Transcribed locus | −5.58 | 0.00002 | 0.00359 | −0.48 | 0.72 | −1.39 | 14 |
| 1371896_at | growth arrest and DNA- | −6.02 | 0.00001 | 0.00189 | −0.43 | 0.74 | −1.35 | 14 |
| | damage-inducible, gamma | | | | | | | |
| | interacting protein 1 (predicted) | | | | | | | |
| 1371918_at | CD99 | −5.35 | 0.00004 | 0.00476 | −0.37 | 0.77 | −1.29 | 14 |
| 1372057_at | CDNA clone MGC: 124976 | −6.12 | 0.00001 | 0.00173 | −0.38 | 0.77 | −1.30 | 14 |
| | IMAGE: 7110947 | | | | | | | |
| 1372137_at | biogenesis of lysosome-related | −6.03 | 0.00001 | 0.00187 | −0.41 | 0.75 | −1.32 | 14 |
| | organelles complex-1, subunit | | | | | | | |
| | 1 (predicted) | | | | | | | |
| 1372142_at | arsA arsenite transporter, ATP- | −4.93 | 0.00009 | 0.00829 | −0.37 | 0.77 | −1.30 | 14 |
| | binding, homolog 1 (bacterial) | | | | | | | |
| | (predicted) | | | | | | | |
| 1372236_at | Similar to Caspase recruitment | −4.90 | 0.00010 | 0.00871 | −0.36 | 0.78 | −1.29 | 14 |
| | domain protein 4 | | | | | | | |
| 1372469_at | Transcribed locus | −4.84 | 0.00011 | 0.00945 | −0.36 | 0.78 | −1.28 | 14 |
| 1372697_at | mitochondrial ribosomal | −5.70 | 0.00002 | 0.00299 | −0.58 | 0.67 | −1.49 | 14 |
| | protein S15 | | | | | | | |
| 1373031_at | tripartite motif protein 8 | −5.13 | 0.00006 | 0.00628 | −0.44 | 0.74 | −1.36 | 14 |
| | (predicted) | | | | | | | |
| 1373105_at | interleukin 1 receptor-like 1 | −5.01 | 0.00008 | 0.00742 | −0.37 | 0.77 | −1.30 | 14 |
| | ligand (predicted) | | | | | | | |
| 1373135_at | similar to hypothetical protein | −5.30 | 0.00004 | 0.00503 | −0.55 | 0.68 | −1.46 | 14 |
| | MGC2744 | | | | | | | |
| 1373206_at | similar to FAD104 (predicted) | 6.73 | 0.00000 | 0.00080 | 0.64 | 1.56 | 1.56 | 14 |
| 1373303_at | similar to mKIAA3013 protein | −5.28 | 0.00004 | 0.00514 | −0.48 | 0.72 | −1.39 | 14 |
| 1373347_at | DMT1-associated protein | −6.18 | 0.00001 | 0.00162 | −0.73 | 0.60 | −1.66 | 14 |
| 1373378_at | ATP/GTP binding protein 1 | 5.39 | 0.00003 | 0.00449 | 0.51 | 1.42 | 1.42 | 14 |
| | (predicted) | | | | | | | |
| 1373804_at | Forkhead box P1 (predicted) | −5.28 | 0.00004 | 0.00518 | −0.59 | 0.66 | −1.51 | 14 |
| 1373885_at | chromobox homolog 5 | −5.94 | 0.00001 | 0.00208 | −1.04 | 0.48 | −2.06 | 14 |
| | (Drosophila HP1a) (predicted) | | | | 1.04 | | | |
| 1374002_at | | −6.78 | 0.00000 | 0.00074 | −0.86 | 0.55 | −1.82 | 14 |
| 1374283_at | fetal Alzheimer antigen | −7.44 | 0.00000 | 0.00032 | −0.74 | 0.60 | −1.67 | 14 |
| | (predicted) | | | | | | | |
| 1374425_at | transducin-like enhancer of | −4.91 | 0.00010 | 0.00849 | −0.40 | 0.76 | −1.32 | 14 |
| | split 1, homolog of Drosophila | | | | | | | |
| | E(spl) (predicted) | | | | | | | |
| 1374509_at | Similar to RIKEN cDNA | −5.62 | 0.00002 | 0.00337 | −0.47 | 0.72 | −1.39 | 14 |
| | 1110018O08 | | | | | | | |
| 1374511_at | | 5.60 | 0.00002 | 0.00345 | 0.55 | 1.47 | 1.47 | 14 |
| 1374657_at | Transcribed locus | −4.88 | 0.00010 | 0.00890 | −0.34 | 0.79 | −1.27 | 14 |
| 1374733_at | symplekin (predicted) | −5.04 | 0.00007 | 0.00716 | −0.36 | 0.78 | −1.28 | 14 |
| 1374772_at | similar to Chromosome 13 | 5.18 | 0.00005 | 0.00581 | 0.46 | 1.38 | 1.38 | 14 |
| | open reading frame 21 | | | | | | | |
| 1374837_at | B-cell CLL/lymphoma 7C | −8.92 | 0.00000 | 0.00006 | −0.71 | 0.61 | −1.63 | 14 |
| | (predicted) | | | | | | | |
| 1374851_at | similar to RIKEN cDNA | −4.89 | 0.00010 | 0.00879 | −0.39 | 0.76 | −1.31 | 14 |
| | 2810405O22 (predicted) | | | | | | | |
| 1374852_at | hypothetical LOC362592 | −5.20 | 0.00005 | 0.00579 | −0.37 | 0.78 | −1.29 | 14 |
| 1375214_at | UDP-N-acetyl-alpha-D- | −5.31 | 0.00004 | 0.00500 | −0.58 | 0.67 | −1.50 | 14 |
| | galactosamine:polypeptide N- | | | | | | | |
| | acetylgalactosaminyltransferase | | | | | | | |
| | 2 (predicted) | | | | | | | |
| 1375335_at | heat shock 90 kDa protein 1, | −5.26 | 0.00004 | 0.00538 | −0.55 | 0.68 | −1.46 | 14 |
| | beta | | | | | | | |
| 1375396_at | pumilio 1 (Drosophila) | −10.05 | 0.00000 | 0.00003 | −0.92 | 0.53 | −1.89 | 14 |
| | (predicted) | | | | | | | |
| 1375421_a_at | praja 2, RING-H2 motif | −6.51 | 0.00000 | 0.00102 | −0.60 | 0.66 | −1.52 | 14 |
| | containing | | | | | | | |
| 1375453_at | | −12.32 | 0.00000 | 0.00000 | −1.02 | 0.49 | −2.02 | 14 |
| 1375469_at | SWI/SNF related, matrix | −7.97 | 0.00000 | 0.00017 | −0.93 | 0.53 | −1.90 | 14 |
| | associated, actin dependent | | | | | | | |
| | regulator of chromatin, | | | | | | | |
| | subfamily a, member 4 | | | | | | | |
| 1375533_at | vestigial like 4 (Drosophila) | −5.30 | 0.00004 | 0.00505 | −0.61 | 0.66 | −1.52 | 14 |
| | (predicted) | | | | | | | |
| 1375548_at | similar to RIKEN cDNA | −5.64 | 0.00002 | 0.00328 | −0.58 | 0.67 | −1.50 | 14 |
| | 4732418C07 (predicted) | | | | | | | |
| 1375621_at | | −7.05 | 0.00000 | 0.00051 | −0.96 | 0.51 | −1.95 | 14 |
| 1375632_at | similar to 60S ribosomal | −4.85 | 0.00011 | 0.00921 | −0.29 | 0.82 | −1.22 | 14 |
| | protein L38 | | | | | | | |
| 1375650_at | bromodomain containing 4 | −6.64 | 0.00000 | 0.00088 | −0.48 | 0.71 | −1.40 | 14 |
| | (predicted) | | | | | | | |
| 1375658_at | Transcribed locus | −5.00 | 0.00008 | 0.00756 | −0.44 | 0.74 | −1.35 | 14 |
| 1375696_at | interferon (alpha and beta) | 4.81 | 0.00012 | 0.00958 | 0.59 | 1.51 | 1.51 | 14 |
| | receptor 1 (predicted) | | | | | | | |
| 1375703_at | myeloid/lymphoid or mixed- | −10.20 | 0.00000 | 0.00003 | −1.02 | 0.49 | −2.03 | 14 |
| | lineage leukemia 5 (trithorax | | | | | | | |
| | homolog, Drosophila) | | | | | | | |
| | (predicted) | | | | | | | |
| 1375706_at | | −5.01 | 0.00008 | 0.00743 | −0.49 | 0.71 | −1.40 | 14 |
| 1375763_at | similar to 2700008B19Rik | −7.08 | 0.00000 | 0.00050 | −0.54 | 0.69 | −1.45 | 14 |
| | protein | | | | | | | |
| 1375958_at | | −5.13 | 0.00006 | 0.00628 | −0.65 | 0.64 | −1.57 | 14 |
| 1376059_at | | 5.33 | 0.00004 | 0.00483 | 0.35 | 1.28 | 1.28 | 14 |
| 1376256_at | WD repeat and FYVE domain | −9.16 | 0.00000 | 0.00005 | −1.10 | 0.47 | −2.15 | 14 |
| | containing 1 (predicted) | | | | | | | |
| 1376299_at | similar to Retinoblastoma- | −9.22 | 0.00000 | 0.00005 | −0.89 | 0.54 | −1.85 | 14 |
| | binding protein 2 (RBBP-2) | | | | | | | |
| 1376450_at | transmembrane protein 5 | −6.26 | 0.00001 | 0.00147 | −0.55 | 0.68 | −1.46 | 14 |
| | (predicted) | | | | | | | |
| 1376523_at | AT rich interactive domain 4A | −5.53 | 0.00002 | 0.00383 | −0.77 | 0.59 | −1.70 | 14 |
| | (Rbp1 like) (predicted) | | | | | | | |
| 1376524_at | hypothetical protein Dd25 | −6.69 | 0.00000 | 0.00082 | −0.66 | 0.63 | −1.58 | 14 |
| 1376532_at | similar to FAD104 (predicted) | 6.06 | 0.00001 | 0.00178 | 0.56 | 1.47 | 1.47 | 14 |
| 1376728_at | Transcribed locus | −4.80 | 0.00012 | 0.00966 | −0.35 | 0.78 | −1.27 | 14 |
| 1376917_at | zinc finger protein 292 | −5.21 | 0.00005 | 0.00571 | −0.66 | 0.63 | −1.58 | 14 |
| 1376982_at | Transcribed locus | −5.49 | 0.00003 | 0.00405 | −0.45 | 0.73 | −1.37 | 14 |
| 1377105_at | | −6.97 | 0.00000 | 0.00056 | −0.89 | 0.54 | −1.85 | 14 |
| 1377302_a_at | methylmalonic aciduria | −5.10 | 0.00006 | 0.00660 | −0.52 | 0.70 | −1.43 | 14 |
| | (cobalamin deficiency) type A | | | | | | | |
| | (predicted) | | | | | | | |
| 1377524_at | similar to CG18661-PA | −5.36 | 0.00003 | 0.00465 | −0.43 | 0.74 | −1.35 | 14 |
| | (predicted) | | | | | | | |
| 1377663_at | ras homolog gene family, | −5.00 | 0.00008 | 0.00756 | −0.87 | 0.55 | −1.82 | 14 |
| | member E | | | | | | | |
| 1377683_at | similar to hypothetical protein | −6.63 | 0.00000 | 0.00088 | −0.56 | 0.68 | −1.47 | 14 |
| | FLJ13045 (predicted) | | | | | | | |
| 1377728_at | LOC499567 | −5.45 | 0.00003 | 0.00419 | −1.03 | 0.49 | −2.04 | 14 |
| 1377766_at | Transcribed locus | 4.80 | 0.00012 | 0.00964 | 0.37 | 1.29 | 1.29 | 14 |
| 1377899_at | similar to RIKEN cDNA | −4.99 | 0.00008 | 0.00760 | −0.46 | 0.73 | −1.38 | 14 |
| | 2810025M15 (predicted) | | | | | | | |
| 1377906_at | DEAH (Asp-Glu-Ala-His) box | −4.82 | 0.00012 | 0.00950 | −0.73 | 0.60 | −1.66 | 14 |
| | polypeptide 36 (predicted) | | | | | | | |
| 1377914_at | serine/arginine repetitive | −6.41 | 0.00000 | 0.00120 | −0.98 | 0.51 | −1.97 | 14 |
| | matrix 1 (predicted) | | | | | | | |
| 1378155_at | similar to KIAA1096 protein | −5.68 | 0.00002 | 0.00313 | −0.89 | 0.54 | −1.86 | 14 |
| 1378163_at | Transcribed locus | −4.86 | 0.00011 | 0.00913 | −0.78 | 0.58 | −1.71 | 14 |
| 1378170_at | Transcribed locus | −5.00 | 0.00008 | 0.00756 | −0.92 | 0.53 | −1.90 | 14 |
| 1378194_a_at | rap2 interacting protein x | −4.82 | 0.00012 | 0.00950 | −0.72 | 0.61 | −1.65 | 14 |
| 1378361_at | chromodomain helicase DNA | −7.32 | 0.00000 | 0.00039 | −0.73 | 0.60 | −1.66 | 14 |
| | binding protein 7 (predicted) | | | | | | | |
| 1378453_at | | −4.84 | 0.00011 | 0.00938 | −0.74 | 0.60 | −1.66 | 14 |
| 1378504_at | Insulin-like growth factor I | −5.41 | 0.00003 | 0.00440 | −0.96 | 0.51 | −1.95 | 14 |
| | mRNA, 3′ end of mRNA | | | | | | | |
| 1378786_at | Transcribed locus, weakly | 4.89 | 0.00010 | 0.00879 | 0.33 | 1.25 | 1.25 | 14 |
| | similar to NP_780607.2 | | | | | | | |
| | hypothetical protein | | | | | | | |
| | LOC109050 [Mus musculus] | | | | | | | |
| 1379062_at | similar to Expressed sequence | −6.60 | 0.00000 | 0.00090 | −1.08 | 0.47 | −2.12 | 14 |
| | AU019823 | | | | | | | |
| 1379073_at | Similar to RIKEN cDNA | −5.51 | 0.00003 | 0.00394 | −0.49 | 0.71 | −1.40 | 14 |
| | 2310067G05 | | | | | | | |
| 1379101_at | DEAH (Asp-Glu-Ala-His) box | −5.55 | 0.00002 | 0.00375 | −0.87 | 0.55 | −1.82 | 14 |
| | polypeptide 36 (predicted) | | | | | | | |
| 1379112_at | AT rich interactive domain 4A | −5.70 | 0.00002 | 0.00299 | −0.44 | 0.74 | −1.35 | 14 |
| | (Rbp1 like) (predicted) | | | | | | | |
| 1379232_at | TBC1D12: TBC1 domain | −6.98 | 0.00000 | 0.00056 | −1.40 | 0.38 | −2.63 | 14 |
| | family, member 12 (predicted) | | | | | | | |
| 1379330_s_at | CDNA clone IMAGE: 7316839 | −4.80 | 0.00012 | 0.00967 | −0.36 | 0.78 | −1.28 | 14 |
| 1379332_at | Transcribed locus, strongly | −4.88 | 0.00010 | 0.00886 | −0.61 | 0.66 | −1.52 | 14 |
| | similar to XP_417265.1 | | | | | | | |
| | PREDICTED: similar to F- | | | | | | | |
| | box-WD40 repeat protein 6 | | | | | | | |
| | [Gallus gallus] | | | | | | | |
| 1379399_at | similar to cDNA sequence | −5.37 | 0.00003 | 0.00459 | −0.42 | 0.75 | −1.34 | 14 |
| | BC016188 (predicted) | | | | | | | |
| 1379457_at | neural precursor cell expressed, | −5.39 | 0.00003 | 0.00449 | −0.56 | 0.68 | −1.48 | 14 |
| | developmentally down- | | | | | | | |
| | regulated gene 1 (predicted) | | | | | | | |
| 1379469_at | similar to transducin (beta)-like | −6.23 | 0.00001 | 0.00153 | −0.91 | 0.53 | −1.88 | 14 |
| | 1 X-linked | | | | | | | |
| 1379485_at | eukaryotic translation initiation | −7.08 | 0.00000 | 0.00050 | −1.68 | 0.31 | −3.21 | 14 |
| | factor 3, subunit 10 (theta) | | | | | | | |
| | (predicted) | | | | | | | |
| 1379571_at | plakophilin 4 (predicted) | −5.42 | 0.00003 | 0.00436 | −0.74 | 0.60 | −1.67 | 14 |
| 1379578_at | similar to Zbtb20 protein | −8.89 | 0.00000 | 0.00006 | −0.71 | 0.61 | −1.63 | 14 |
| 1379662_a_at | SNF related kinase | 4.93 | 0.00009 | 0.00829 | 0.36 | 1.29 | 1.29 | 14 |
| 1379715_at | similar to CG9346-PA | −4.93 | 0.00009 | 0.00829 | −0.71 | 0.61 | −1.63 | 14 |
| | (predicted) | | | | | | | |
| 1379826_at | similar to hypothetical protein | −5.95 | 0.00001 | 0.00208 | −0.62 | 0.65 | −1.54 | 14 |
| | MGC31967 | | | | | | | |
| 1380008_at | similar to Neurofilament triplet | −5.11 | 0.00006 | 0.00645 | −0.60 | 0.66 | −1.52 | 14 |
| | H protein (200 kDa | | | | | | | |
| | neurofilament protein) | | | | | | | |
| | (Neurofilament heavy | | | | | | | |
| | polypeptide) (NF-H) | | | | | | | |
| | (predicted) | | | | | | | |
| 1380060_at | DNA topoisomerase I, | −5.23 | 0.00005 | 0.00566 | −0.53 | 0.69 | −1.44 | 14 |
| | mitochondrial | | | | | | | |
| 1380062_at | membrane protein, | −6.88 | 0.00000 | 0.00065 | −0.75 | 0.59 | −1.68 | 14 |
| | palmitoylated 6 (MAGUK p55 | | | | | | | |
| | subfamily member 6) | | | | | | | |
| | (predicted) | | | | | | | |
| 1380166_at | Similar to hypothetical protein | 5.63 | 0.00002 | 0.00333 | 0.34 | 1.27 | 1.27 | 14 |
| | FLJ12056 | | | | | | | |
| 1380371_at | delangin (predicted) | −9.37 | 0.00000 | 0.00005 | −0.94 | 0.52 | −1.91 | 14 |
| 1380446_at | myeloid/lymphoid or mixed- | −5.00 | 0.00008 | 0.00756 | −0.62 | 0.65 | −1.54 | 14 |
| | lineage leukemia (trithorax | | | | | | | |
| | homolog, Drosophila); | | | | | | | |
| | translocated to, 10 (predicted) | | | | | | | |
| 1380503_at | hypothetical LOC305452 | −6.07 | 0.00001 | 0.00178 | −0.62 | 0.65 | −1.53 | 14 |
| | (predicted) | | | | | | | |
| 1380728_at | Similar to collapsin response | 6.09 | 0.00001 | 0.00178 | 0.49 | 1.41 | 1.41 | 14 |
| | mediator protein-2A | | | | | | | |
| 1381469_a_at | PERQ amino acid rich, with | −5.49 | 0.00003 | 0.00405 | −0.51 | 0.70 | −1.43 | 14 |
| | GYF domain 1 (predicted) | | | | | | | |
| 1381525_at | | −4.82 | 0.00012 | 0.00952 | −0.41 | 0.75 | −1.33 | 14 |
| 1381542_at | UBX domain containing 2 | −6.15 | 0.00001 | 0.00171 | −0.83 | 0.56 | −1.78 | 14 |
| | (predicted) | | | | | | | |
| 1381548_at | golgi phosphoprotein 4 | −5.81 | 0.00001 | 0.00256 | −0.69 | 0.62 | −1.61 | 14 |
| | (predicted) | | | | | | | |
| 1381567_at | hypothetical LOC294390 | 4.97 | 0.00008 | 0.00800 | 0.36 | 1.29 | 1.29 | 14 |
| | (predicted) | | | | | | | |
| 1381764_s_at | ring finger protein 126 | −5.54 | 0.00002 | 0.00382 | −0.51 | 0.70 | −1.42 | 14 |
| | (predicted) | | | | | | | |
| 1381809_at | ankyrin repeat domain 11 | −5.94 | 0.00001 | 0.00209 | −1.11 | 0.46 | −2.17 | 14 |
| | (predicted) | | | | | | | |
| 1381829_at | | −6.27 | 0.00000 | 0.00145 | −1.07 | 0.48 | −2.10 | 14 |
| 1381878_at | ubinuclein 1 (predicted) | −5.82 | 0.00001 | 0.00252 | −1.18 | 0.44 | −2.26 | 14 |
| 1381958_at | similar to mKIAA0259 protein | −6.90 | 0.00000 | 0.00062 | −1.27 | 0.41 | −2.42 | 14 |
| 1382000_at | | 4.82 | 0.00012 | 0.00950 | 0.41 | 1.33 | 1.33 | 14 |
| 1382009_at | Transcribed locus | −5.39 | 0.00003 | 0.00449 | −0.69 | 0.62 | −1.62 | 14 |
| 1382027_at | LOC498010 | −6.28 | 0.00000 | 0.00144 | −0.76 | 0.59 | −1.70 | 14 |
| 1382056_at | similar to splicing factor p54 | −8.13 | 0.00000 | 0.00016 | −0.97 | 0.51 | −1.96 | 14 |
| 1382109_at | nuclear NF-kappaB activating | −5.83 | 0.00001 | 0.00250 | −0.62 | 0.65 | −1.53 | 14 |
| | protein | | | | | | | |
| 1382155_at | | 6.37 | 0.00000 | 0.00126 | 0.58 | 1.50 | 1.50 | 14 |
| 1382193_at | Transcribed locus | −6.07 | 0.00001 | 0.00178 | −1.42 | 0.37 | −2.67 | 14 |
| 1382306_at | Ariadne ubiquitin-conjugating | 6.59 | 0.00000 | 0.00090 | 0.59 | 1.50 | 1.50 | 14 |
| | enzyme E2 binding protein | | | | | | | |
| | homolog 1 (Drosophila) | | | | | | | |
| | (predicted) | | | | | | | |
| 1382307_at | protein phosphatase 1, | −4.79 | 0.00013 | 0.00976 | −0.47 | 0.72 | −1.39 | 14 |
| | regulatory (inhibitor) subunit | | | | | | | |
| | 12A | | | | | | | |
| 1382358_at | Similar to SRY (sex | −5.34 | 0.00004 | 0.00482 | −0.65 | 0.64 | −1.57 | 14 |
| | determining region Y)-box 5 | | | | | | | |
| | isoform a | | | | | | | |
| 1382372_at | Aryl hydrocarbon receptor | −5.07 | 0.00007 | 0.00680 | −0.74 | 0.60 | −1.67 | 14 |
| 1382430_at | similar to KIAA1585 protein | −5.62 | 0.00002 | 0.00338 | −0.58 | 0.67 | −1.50 | 14 |
| | (predicted) | | | | | | | |
| 1382434_at | ectonucleoside triphosphate | −5.89 | 0.00001 | 0.00229 | −0.73 | 0.60 | −1.66 | 14 |
| | diphosphohydrolase 5 | | | | | | | |
| 1382466_at | similar to RIKEN cDNA | −5.43 | 0.00003 | 0.00433 | −0.98 | 0.51 | −1.97 | 14 |
| | 6530403A03 (predicted) | | | | | | | |
| 1382551_at | similar to Intersectin 2 (SH3 | −6.72 | 0.00000 | 0.00080 | −1.41 | 0.38 | −2.67 | 14 |
| | domain-containing protein 1B) | | | | | | | |
| | (SH3P18) (SH3P18-like | | | | | | | |
| | WASP associated protein) | | | | | | | |
| 1382558_at | transcription factor 3 | −6.13 | 0.00001 | 0.00171 | −0.62 | 0.65 | −1.54 | 14 |
| | (predicted) | | | | | | | |
| 1382573_at | Transcribed locus | 5.08 | 0.00007 | 0.00677 | 0.38 | 1.30 | 1.30 | 14 |
| 1382584_at | similar to mKIAA1321 protein | −7.22 | 0.00000 | 0.00042 | −1.15 | 0.45 | −2.22 | 14 |
| 1382620_at | ankyrin repeat domain 11 | −9.69 | 0.00000 | 0.00003 | −0.95 | 0.52 | −1.93 | 14 |
| | (predicted) | | | | | | | |
| 1382797_at | similar to 1500019C06Rik | −5.02 | 0.00008 | 0.00742 | −0.47 | 0.72 | −1.39 | 14 |
| | protein | | | | | | | |
| 1382813_at | similar to RIKEN cDNA | −5.36 | 0.00004 | 0.00467 | −0.46 | 0.73 | −1.38 | 14 |
| | 4930444A02 (predicted) | | | | | | | |
| 1382862_at | Transcribed locus | −6.23 | 0.00001 | 0.00153 | −1.16 | 0.45 | −2.23 | 14 |
| 1382904_at | similar to hypothetical protein | −9.04 | 0.00000 | 0.00005 | −0.85 | 0.56 | −1.80 | 14 |
| | DKFZp434K1421 (predicted) | | | | | | | |
| 1382935_at | similar to Hypothetical protein | −6.54 | 0.00000 | 0.00097 | −0.64 | 0.64 | −1.56 | 14 |
| | KIAA0141 | | | | | | | |
| 1382939_at | translocated promoter region | −5.18 | 0.00005 | 0.00581 | −1.13 | 0.46 | −2.19 | 14 |
| | (predicted) | | | | | | | |
| 1382957_at | similar to cisplatin resistance- | −8.04 | 0.00000 | 0.00016 | −1.08 | 0.47 | −2.11 | 14 |
| | associated overexpressed | | | | | | | |
| | protein (predicted) | | | | | | | |
| 1382960_at | Transcribed locus | −5.95 | 0.00001 | 0.00208 | −0.77 | 0.59 | −1.70 | 14 |
| 1382972_at | Transcribed locus, strongly | 5.17 | 0.00005 | 0.00595 | 0.37 | 1.29 | 1.29 | 14 |
| | similar to XP_226713.2 | | | | | | | |
| | PREDICTED: similar to Src- | | | | | | | |
| | associated protein SAW | | | | | | | |
| | [Rattus norvegicus] | | | | | | | |
| 1383008_at | SMC4 structural maintenance | −5.19 | 0.00005 | 0.00581 | −0.98 | 0.51 | −1.97 | 14 |
| | of chromosomes 4-like 1 | | | | | | | |
| | (yeast) (predicted) | | | | | | | |
| 1383040_a_at | | −5.46 | 0.00003 | 0.00419 | −0.47 | 0.72 | −1.38 | 14 |
| 1383052_a_at | | −6.54 | 0.00000 | 0.00097 | −0.62 | 0.65 | −1.53 | 14 |
| 1383054_at | | −7.86 | 0.00000 | 0.00019 | −0.76 | 0.59 | −1.70 | 14 |
| 1383060_at | G kinase anchoring protein 1 | −5.82 | 0.00001 | 0.00255 | −0.44 | 0.74 | −1.35 | 14 |
| | (predicted) | | | | | | | |
| 1383085_at | Similar to Sh3bgrl protein | −5.20 | 0.00005 | 0.00576 | −0.86 | 0.55 | −1.81 | 14 |
| 1383179_at | Similar to hypothetical protein | −5.10 | 0.00006 | 0.00660 | −0.75 | 0.60 | −1.68 | 14 |
| | HSPC129 (predicted) | | | | | | | |
| 1383184_at | zinc and ring finger 1 | 5.03 | 0.00007 | 0.00731 | 0.39 | 1.31 | 1.31 | 14 |
| | (predicted) | | | | | | | |
| 1383334_at | Transcribed locus | −5.37 | 0.00003 | 0.00461 | −0.46 | 0.73 | −1.37 | 14 |
| 1383455_at | glutamyl-prolyl-tRNA | −6.80 | 0.00000 | 0.00072 | −0.72 | 0.61 | −1.65 | 14 |
| | synthetase (predicted) | | | | | | | |
| 1383535_at | ankyrin repeat and SOCS box- | 6.24 | 0.00001 | 0.00152 | 0.38 | 1.30 | 1.30 | 14 |
| | containing protein 8 (predicted) | | | | | | | |
| 1383615_a_at | similar to HECT domain | −6.06 | 0.00001 | 0.00178 | −1.08 | 0.47 | −2.11 | 14 |
| | containing 1 | | | | | | | |
| 1383687_at | | −5.40 | 0.00003 | 0.00441 | −0.43 | 0.74 | −1.34 | 14 |
| 1383776_at | Transcribed locus | 6.41 | 0.00000 | 0.00120 | 0.62 | 1.54 | 1.54 | 14 |
| 1383786_at | Transcribed locus | −5.13 | 0.00006 | 0.00628 | −0.50 | 0.71 | −1.41 | 14 |
| 1383825_at | radixin | −9.20 | 0.00000 | 0.00005 | −1.01 | 0.50 | −2.01 | 14 |
| 1383827_at | tousled-like kinase 1 | −6.09 | 0.00001 | 0.00178 | −1.25 | 0.42 | −2.37 | 14 |
| | (predicted) | | | | | | | |
| 1384125_at | myeloid/lymphoid or mixed- | −5.97 | 0.00001 | 0.00202 | −0.51 | 0.70 | −1.42 | 14 |
| | lineage leukemia 5 (trithorax | | | | | | | |
| | homolog, Drosophila) | | | | | | | |
| | (predicted) | | | | | | | |
| 1384131_at | ADP-ribosylation factor-like 6 | 6.10 | 0.00001 | 0.00176 | 0.70 | 1.63 | 1.63 | 14 |
| | interacting protein 2 (predicted) | | | | | | | |
| 1384146_at | Similar to CD69 antigen (p60, | −5.22 | 0.00005 | 0.00568 | −1.35 | 0.39 | −2.55 | 14 |
| | early T-cell activation antigen) | | | | | | | |
| 1384154_at | WW domain binding protein 4 | −5.33 | 0.00004 | 0.00483 | −0.52 | 0.70 | −1.43 | 14 |
| 1384260_at | Transcribed locus | −6.36 | 0.00000 | 0.00126 | −0.66 | 0.63 | −1.58 | 14 |
| 1384263_at | ATP-binding cassette, sub- | −7.27 | 0.00000 | 0.00040 | −0.72 | 0.61 | −1.64 | 14 |
| | family A (ABC1), member 13 | | | | | | | |
| | (predicted) | | | | | | | |
| | similar to hypothetical protein | | | | | | | |
| | MGC33214 (predicted) | | | | | | | |
| 1384339_s_at | casein kinase II, alpha 1 | −8.81 | 0.00000 | 0.00006 | −1.83 | 0.28 | −3.56 | 14 |
| | polypeptide | | | | | | | |
| 1384376_at | similar to FLJ14281 protein | −5.42 | 0.00003 | 0.00436 | −0.65 | 0.64 | −1.57 | 14 |
| 1384394_at | | −7.21 | 0.00000 | 0.00042 | −0.61 | 0.65 | −1.53 | 14 |
| 1384609_a_at | similar to RIKEN cDNA | −6.61 | 0.00000 | 0.00090 | −0.91 | 0.53 | −1.87 | 14 |
| | B230380D07 (predicted) | | | | | | | |
| 1384766_a_at | similar to PHD finger protein | −5.18 | 0.00005 | 0.00581 | −0.70 | 0.61 | −1.63 | 14 |
| | 14 isoform 1 | | | | | | | |
| 1384791_at | UDP-GlcNAc:betaGal beta- | −5.08 | 0.00006 | 0.00671 | −0.75 | 0.60 | −1.68 | 14 |
| | 1,3-N- | | | | | | | |
| | acetylglucosaminyltransferase | | | | | | | |
| | 1 (predicted) | | | | | | | |
| 1384792_at | formin binding protein 3 | −6.70 | 0.00000 | 0.00082 | −0.97 | 0.51 | −1.96 | 14 |
| | (predicted) | | | | | | | |
| 1384857_at | A kinase (PRKA) anchor | −5.62 | 0.00002 | 0.00338 | −1.05 | 0.48 | −2.07 | 14 |
| | protein (yotiao) 9 | | | | | | | |
| 1385006_at | alpha thalassemia/mental | −4.94 | 0.00009 | 0.00829 | −0.45 | 0.73 | −1.37 | 14 |
| | retardation syndrome X-linked | | | | | | | |
| | homolog (human) | | | | | | | |
| 1385038_at | similar to hedgehog-interacting | −9.94 | 0.00000 | 0.00003 | −0.80 | 0.57 | −1.75 | 14 |
| | protein | | | | | | | |
| 1385076_at | | −5.78 | 0.00001 | 0.00271 | −0.57 | 0.68 | −1.48 | 14 |
| 1385077_at | similar to golgi-specific | −7.83 | 0.00000 | 0.00019 | −1.05 | 0.48 | −2.07 | 14 |
| | brefeldin A-resistance guanine | | | | | | | |
| | nucleotide exchange factor 1 | | | | | | | |
| | (predicted) | | | | | | | |
| 1385101_a_at | Unknown (protein for | −5.53 | 0.00002 | 0.00383 | −0.97 | 0.51 | −1.96 | 14 |
| | MGC: 73017) | | | | | | | |
| 1385108_at | Transcribed locus | −4.83 | 0.00011 | 0.00946 | −1.27 | 0.42 | −2.40 | 14 |
| 1385240_at | WD repeat domain 33 | −4.81 | 0.00012 | 0.00958 | −0.93 | 0.52 | −1.91 | 14 |
| | (predicted) | | | | | | | |
| 1385320_at | similar to Pdz-containing | −5.26 | 0.00004 | 0.00530 | −0.47 | 0.72 | −1.38 | 14 |
| | protein | | | | | | | |
| 1385407_at | TCDD-inducible poly(ADP- | −5.46 | 0.00003 | 0.00417 | −1.34 | 0.39 | −2.54 | 14 |
| | ribose) polymerase (predicted) | | | | | | | |
| 1385408_at | similar to mKIAA0518 protein | −5.86 | 0.00001 | 0.00236 | −1.31 | 0.40 | −2.49 | 14 |
| 1385689_at | Transcribed locus | −4.83 | 0.00011 | 0.00948 | −0.68 | 0.62 | −1.60 | 14 |
| 1385852_at | CREB binding protein | −4.85 | 0.00011 | 0.00927 | −0.53 | 0.69 | −1.44 | 14 |
| | hypothetical gene supported by | | | | | | | |
| | NM_133381 | | | | | | | |
| 1385931_at | hook homolog 3 | −7.30 | 0.00000 | 0.00040 | −1.66 | 0.32 | −3.16 | 14 |
| 1385999_at | YME1-like 1 (S. cerevisiae) | −4.80 | 0.00012 | 0.00964 | −0.63 | 0.64 | −1.55 | 14 |
| 1386191_a_at | Transcribed locus | 5.22 | 0.00005 | 0.00568 | 0.46 | 1.37 | 1.37 | 14 |
| 1386641_at | Transcribed locus | −5.41 | 0.00003 | 0.00440 | −0.97 | 0.51 | −1.95 | 14 |
| 1386793_at | similar to zinc finger protein 61 | −5.28 | 0.00004 | 0.00514 | −0.59 | 0.66 | −1.51 | 14 |
| 1387087_at | CCAAT/enhancer binding | −5.43 | 0.00003 | 0.00435 | −0.61 | 0.66 | −1.52 | 14 |
| | protein (C/EBP), beta | | | | | | | |
| 1387306_a_at | early growth response 2 | 4.82 | 0.00012 | 0.00950 | 0.33 | 1.26 | 1.26 | 14 |
| 1387365_at | nuclear receptor subfamily 1, | −4.86 | 0.00011 | 0.00913 | −0.35 | 0.78 | −1.27 | 14 |
| | group H, member 3 | | | | | | | |
| 1387415_a_at | syntaxin binding protein 5 | 4.86 | 0.00011 | 0.00913 | 0.40 | 1.32 | 1.32 | 14 |
| | (tomosyn) | | | | | | | |
| 1387458_at | ring finger protein 4 | 7.05 | 0.00000 | 0.00051 | 0.75 | 1.69 | 1.69 | 14 |
| 1387664_at | ATPase, H+ transporting, V1 | 5.55 | 0.00002 | 0.00375 | 0.46 | 1.38 | 1.38 | 14 |
| | subunit B, isoform 2 | | | | | | | |
| 1387757_at | liver regeneration p-53 related | 5.19 | 0.00005 | 0.00581 | 0.51 | 1.42 | 1.42 | 14 |
| | protein | | | | | | | |
| 1387760_a_at | one cut domain, family | −6.12 | 0.00001 | 0.00171 | −1.58 | 0.34 | −2.98 | 14 |
| | member 1 | | | | | | | |
| 1387789_at | v-ets erythroblastosis virus E26 | −6.61 | 0.00000 | 0.00090 | −0.58 | 0.67 | −1.50 | 14 |
| | oncogene like (avian) | | | | | | | |
| 1387915_at | Ratsg2 | −4.79 | 0.00013 | 0.00976 | −0.33 | 0.79 | −1.26 | 14 |
| 1387947_at | v-maf musculoaponeurotic | −5.08 | 0.00007 | 0.00674 | −0.80 | 0.57 | −1.74 | 14 |
| | fibrosarcoma oncogene family, | | | | | | | |
| | protein B (avian) | | | | | | | |
| 1388022_a_at | dynamin 1-like | 4.95 | 0.00009 | 0.00811 | 0.43 | 1.35 | 1.35 | 14 |
| 1388059_a_at | solute carrier family 11 | 5.75 | 0.00001 | 0.00280 | 0.43 | 1.35 | 1.35 | 14 |
| | (proton-coupled divalent metal | | | | | | | |
| | ion transporters), member 2 | | | | | | | |
| 1388089_a_at | ring finger protein 4 | 5.73 | 0.00002 | 0.00289 | 0.50 | 1.41 | 1.41 | 14 |
| 1388157_at | myristoylated alanine rich | −5.76 | 0.00001 | 0.00276 | −0.51 | 0.70 | −1.43 | 14 |
| | protein kinase C substrate | | | | | | | |
| 1388196_at | NCK-associated protein 1 | 5.31 | 0.00004 | 0.00500 | 0.48 | 1.40 | 1.40 | 14 |
| 1388251_at | protein kinase C, lambda | 5.21 | 0.00005 | 0.00571 | 0.55 | 1.47 | 1.47 | 14 |
| 1388313_at | ribosomal protein s25 | −4.83 | 0.00011 | 0.00946 | −0.63 | 0.65 | −1.54 | 14 |
| 1388353_at | proliferation-associated 2G4, | −6.35 | 0.00000 | 0.00127 | −0.67 | 0.63 | −1.59 | 14 |
| | 38 kDa | | | | | | | |
| 1388388_at | Protein phosphatase 2, | −5.32 | 0.00004 | 0.00487 | −0.41 | 0.75 | −1.33 | 14 |
| | regulatory subunit B (B56), | | | | | | | |
| | delta isoform (predicted) | | | | | | | |
| 1388396_at | serine/threonine kinase 25 | −5.49 | 0.00003 | 0.00404 | −0.34 | 0.79 | −1.27 | 14 |
| | (STE20 homolog, yeast) | | | | | | | |
| 1388503_at | similar to CREBBP/EP300 | −6.06 | 0.00001 | 0.00178 | −0.40 | 0.76 | −1.32 | 14 |
| | inhibitory protein 1 | | | | | | | |
| 1388714_at | elongation factor RNA | −5.88 | 0.00001 | 0.00229 | −0.46 | 0.73 | −1.37 | 14 |
| | polymerase II (predicted) | | | | | | | |
| 1388735_at | Similar to keratin associated | 4.84 | 0.00011 | 0.00945 | 0.50 | 1.41 | 1.41 | 14 |
| | protein 10-6 | | | | | | | |
| 1388752_at | BCL2-associated transcription | −4.81 | 0.00012 | 0.00958 | −0.40 | 0.76 | −1.32 | 14 |
| | factor 1 (predicted) | | | | | | | |
| 1388849_at | Protease, serine, 25 (predicted) | −5.97 | 0.00001 | 0.00202 | −0.50 | 0.71 | −1.41 | 14 |
| 1388888_at | Transcribed locus | 5.13 | 0.00006 | 0.00632 | 0.40 | 1.32 | 1.32 | 14 |
| 1389268_at | similar to DNA polymerase | −5.21 | 0.00005 | 0.00571 | −0.37 | 0.77 | −1.29 | 14 |
| | lambda | | | | | | | |
| 1389307_at | similar to Amyloid beta (A4) | −4.91 | 0.00010 | 0.00854 | −0.49 | 0.71 | −1.40 | 14 |
| | precursor-like protein 1 | | | | | | | |
| 1389419_at | Transcribed locus | −6.54 | 0.00000 | 0.00097 | −1.30 | 0.40 | −2.47 | 14 |
| 1389432_at | pre-B-cell leukemia | −4.93 | 0.00009 | 0.00829 | −0.55 | 0.69 | −1.46 | 14 |
| | transcription factor 2 | | | | | | | |
| 1389444_at | Transcribed locus | −6.84 | 0.00000 | 0.00068 | −1.06 | 0.48 | −2.09 | 14 |
| 1389806_at | Transcribed locus | −7.63 | 0.00000 | 0.00026 | −0.54 | 0.69 | −1.46 | 14 |
| 1389868_at | similar to RCK | −6.34 | 0.00000 | 0.00128 | −1.47 | 0.36 | −2.76 | 14 |
| 1389963_at | P55 mRNA for p55 protein | −5.54 | 0.00002 | 0.00378 | −0.44 | 0.74 | −1.35 | 14 |
| 1389986_at | LOC499304 | −5.39 | 0.00003 | 0.00449 | −2.57 | 0.17 | −5.93 | 14 |
| 1389989_at | alpha thalassemia/mental | −4.93 | 0.00009 | 0.00829 | −0.54 | 0.69 | −1.45 | 14 |
| | retardation syndrome X-linked | | | | | | | |
| | homolog (human) | | | | | | | |
| 1389998_at | Nuclear receptor subfamily 2, | −6.03 | 0.00001 | 0.00187 | −0.69 | 0.62 | −1.61 | 14 |
| | group F, member 2 | | | | | | | |
| 1390048_at | serine/arginine repetitive | −5.81 | 0.00001 | 0.00256 | −1.04 | 0.49 | −2.05 | 14 |
| | matrix 2 (predicted) | | | | | | | |
| 1390120_a_at | ring finger protein 1 | −5.70 | 0.00002 | 0.00299 | −0.36 | 0.78 | −1.28 | 14 |
| 1390121_at | GLIS family zinc finger 2 | 4.81 | 0.00012 | 0.00958 | 0.43 | 1.34 | 1.34 | 14 |
| | (predicted) | | | | | | | |
| 1390227_at | CDNA clone IMAGE: 7300848 | −5.91 | 0.00001 | 0.00219 | −1.03 | 0.49 | −2.04 | 14 |
| 1390360_a_at | similar to Safb2 protein | −4.79 | 0.00013 | 0.00976 | −0.48 | 0.72 | −1.39 | 14 |
| 1390410_at | Transcribed locus | −4.79 | 0.00012 | 0.00973 | −0.49 | 0.71 | −1.40 | 14 |
| 1390436_at | Autophagy 7-like (S. cerevisiae) | −7.88 | 0.00000 | 0.00019 | −1.46 | 0.36 | −2.76 | 14 |
| | (predicted) | | | | | | | |
| 1390448_at | similar to 1110065L07Rik | 5.08 | 0.00007 | 0.00674 | 0.32 | 1.25 | 1.25 | 14 |
| | protein (predicted) | | | | | | | |
| 1390454_at | 4-nitrophenylphosphatase | −5.47 | 0.00003 | 0.00415 | −0.41 | 0.75 | −1.33 | 14 |
| | domain and non-neuronal | | | | | | | |
| | SNAP25-like protein homolog | | | | | | | |
| | 1 (C. elegans) (predicted) | | | | | | | |
| 1390576_at | Transcribed locus | −5.10 | 0.00006 | 0.00660 | −0.67 | 0.63 | −1.59 | 14 |
| 1390660_at | T-box 2 (predicted) | 5.01 | 0.00008 | 0.00752 | 0.40 | 1.32 | 1.32 | 14 |
| 1390706_at | spectrin beta 2 | −5.55 | 0.00002 | 0.00376 | −0.71 | 0.61 | −1.64 | 14 |
| 1390739_at | similar to zinc finger protein | −5.51 | 0.00003 | 0.00395 | −0.52 | 0.70 | −1.43 | 14 |
| | 609 | | | | | | | |
| | similar to zinc finger protein | | | | | | | |
| | 609 | | | | | | | |
| 1390777_at | sterol-C5-desaturase (fungal | −6.59 | 0.00000 | 0.00090 | −0.70 | 0.61 | −1.63 | 14 |
| | ERG3, delta-5-desaturase) | | | | | | | |
| | homolog (S. cerevisae) | | | | | | | |
| 1390779_at | Similar to phosphoseryl-tRNA | −4.86 | 0.00011 | 0.00913 | −0.64 | 0.64 | −1.56 | 14 |
| | kinase | | | | | | | |
| 1390813_at | Similar to RNA-binding | −5.19 | 0.00005 | 0.00581 | −0.62 | 0.65 | −1.54 | 14 |
| | protein Musashi2-S | | | | | | | |
| 1390884_a_at | UDP-GlcNAc:betaGal beta- | 4.87 | 0.00010 | 0.00904 | 0.49 | 1.40 | 1.40 | 14 |
| | 1,3-N- | | | | | | | |
| | acetylglucosaminyltransferase | | | | | | | |
| | 7 (predicted) | | | | | | | |
| 1391021_at | similar to KIAA1749 protein | −7.55 | 0.00000 | 0.00027 | −0.74 | 0.60 | −1.67 | 14 |
| | (predicted) | | | | | | | |
| 1391170_at | similar to mKIAA1757 protein | −9.21 | 0.00000 | 0.00005 | −2.01 | 0.25 | −4.04 | 14 |
| | (predicted) | | | | | | | |
| 1391222_at | similar to Nedd4 binding | −5.91 | 0.00001 | 0.00219 | −0.81 | 0.57 | −1.76 | 14 |
| | protein 1 (predicted) | | | | | | | |
| 1391297_at | REST corepressor 1 (predicted) | −5.17 | 0.00005 | 0.00595 | −0.92 | 0.53 | −1.89 | 14 |
| 1391578_at | | −8.48 | 0.00000 | 0.00009 | −1.11 | 0.46 | −2.15 | 14 |
| 1391584_at | Transcribed locus | 6.04 | 0.00001 | 0.00185 | 0.45 | 1.37 | 1.37 | 14 |
| 1391625_at | Wiskott-Aldrich syndrome-like | −10.52 | 0.00000 | 0.00002 | −1.28 | 0.41 | −2.43 | 14 |
| | (human) | | | | | | | |
| 1391669_at | protein tyrosine phosphatase, | −6.21 | 0.00001 | 0.00156 | −0.82 | 0.56 | −1.77 | 14 |
| | receptor type, B (predicted) | | | | | | | |
| 1391689_at | similar to Retinoblastoma- | −9.06 | 0.00000 | 0.00005 | −1.20 | 0.44 | −2.30 | 14 |
| | binding protein 2 (RBBP-2) | | | | | | | |
| 1391701_at | MYST histone | −5.18 | 0.00005 | 0.00581 | −0.98 | 0.51 | −1.97 | 14 |
| | acetyltransferase (monocytic | | | | | | | |
| | leukemia) 3 (predicted) | | | | | | | |
| 1391743_at | ELAV (embryonic lethal, | −4.80 | 0.00012 | 0.00964 | −1.36 | 0.39 | −2.58 | 14 |
| | abnormal vision, Drosophila)- | | | | | | | |
| | like 1 (Hu antigen R) | | | | | | | |
| | (predicted) | | | | | | | |
| 1391830_at | copine VIII (predicted) | −5.22 | 0.00005 | 0.00568 | −1.08 | 0.47 | −2.12 | 14 |
| 1391838_at | ankyrin repeat domain 11 | −7.81 | 0.00000 | 0.00019 | −1.15 | 0.45 | −2.23 | 14 |
| | (predicted) | | | | | | | |
| 1391848_at | RNA binding motif protein 27 | −7.02 | 0.00000 | 0.00053 | −0.76 | 0.59 | −1.70 | 14 |
| | (predicted) | | | | | | | |
| 1391968_at | Similar to expressed sequence | −4.89 | 0.00010 | 0.00883 | −0.69 | 0.62 | −1.62 | 14 |
| | AA415817 | | | | | | | |
| 1392000_at | Similar to PHD finger protein | 5.01 | 0.00008 | 0.00742 | 0.45 | 1.37 | 1.37 | 14 |
| | 14 isoform 1 | | | | | | | |
| 1392061_at | minichromosome maintenance | 5.34 | 0.00004 | 0.00482 | 0.54 | 1.46 | 1.46 | 14 |
| | deficient 10 (S. cerevisiae) | | | | | | | |
| | (predicted) | | | | | | | |
| 1392269_at | transcriptional regulator, | −6.23 | 0.00001 | 0.00153 | −1.13 | 0.46 | −2.19 | 14 |
| | SIN3A (yeast) (predicted) | | | | | | | |
| 1392277_at | | −7.29 | 0.00000 | 0.00040 | −0.48 | 0.72 | −1.40 | 14 |
| 1392322_at | GTPase, IMAP family member 7 | −4.83 | 0.00012 | 0.00948 | −0.29 | 0.82 | −1.22 | 14 |
| 1392472_at | similar to myocyte enhancer | −9.77 | 0.00000 | 0.00003 | −0.88 | 0.54 | −1.84 | 14 |
| | factor 2C | | | | | | | |
| 1392552_at | similar to transcription | −6.15 | 0.00001 | 0.00169 | −0.96 | 0.51 | −1.95 | 14 |
| | repressor p66 (predicted) | | | | | | | |
| 1392564_at | myeloid/lymphoid or mixed- | −6.13 | 0.00001 | 0.00171 | −0.57 | 0.68 | −1.48 | 14 |
| | lineage leukemia 5 (trithorax | | | | | | | |
| | homolog, Drosophila) | | | | | | | |
| | (predicted) | | | | | | | |
| 1392629_a_at | similar to MADP-1 protein | −4.93 | 0.00009 | 0.00829 | −0.82 | 0.57 | −1.77 | 14 |
| | (predicted) | | | | | | | |
| 1392738_at | similar to KIAA1096 protein | −5.88 | 0.00001 | 0.00231 | −0.75 | 0.59 | −1.68 | 14 |
| 1392825_at | LOC499256 | −5.20 | 0.00005 | 0.00580 | −0.93 | 0.53 | −1.90 | 14 |
| 1392864_at | Rho GTPase activating protein | −8.05 | 0.00000 | 0.00016 | −1.37 | 0.39 | −2.58 | 14 |
| | 5 (predicted) | | | | | | | |
| 1392932_at | leukocyte receptor cluster | −4.81 | 0.00012 | 0.00958 | −0.79 | 0.58 | −1.73 | 14 |
| | (LRC) member 8 (predicted) | | | | | | | |
| 1392936_at | similar to RNA binding motif | −4.82 | 0.00012 | 0.00950 | −0.88 | 0.54 | −1.85 | 14 |
| | protein 25 | | | | | | | |
| 1392984_at | copine III (predicted) | −7.83 | 0.00000 | 0.00019 | −0.95 | 0.52 | −1.93 | 14 |
| 1393151_at | | 5.03 | 0.00007 | 0.00726 | 0.65 | 1.57 | 1.57 | 14 |
| 1393226_at | Transcribed locus | −4.94 | 0.00009 | 0.00828 | −0.73 | 0.60 | −1.66 | 14 |
| 1393290_at | similar to myocyte enhancer | −5.65 | 0.00002 | 0.00327 | −0.50 | 0.71 | −1.42 | 14 |
| | factor 2C | | | | | | | |
| 1393322_at | TAF15 RNA polymerase II, | −6.18 | 0.00001 | 0.00162 | −1.00 | 0.50 | −2.00 | 14 |
| | TATA box binding protein | | | | | | | |
| | (TBP)-associated factor | | | | | | | |
| | (predicted) | | | | | | | |
| 1393378_at | | −5.72 | 0.00002 | 0.00293 | −0.52 | 0.70 | −1.43 | 14 |
| 1393443_a_at | similar to CGI-112 protein | −5.33 | 0.00004 | 0.00483 | −0.47 | 0.72 | −1.39 | 14 |
| | (predicted) | | | | | | | |
| 1393505_x_at | similar to RIKEN cDNA | −7.60 | 0.00000 | 0.00026 | −0.69 | 0.62 | −1.61 | 14 |
| | B230380D07 (predicted) | | | | | | | |
| 1393511_at | similar to galactose-3-O- | 5.10 | 0.00006 | 0.00655 | 0.41 | 1.33 | 1.33 | 14 |
| | sulfotransferase 4 | | | | | | | |
| 1393560_at | | −4.91 | 0.00010 | 0.00852 | −0.51 | 0.70 | −1.42 | 14 |
| 1393576_at | Transcribed locus | −4.82 | 0.00012 | 0.00950 | −0.62 | 0.65 | −1.54 | 14 |
| 1393593_at | similar to KIAA0597 protein | 5.43 | 0.00003 | 0.00435 | 0.57 | 1.48 | 1.48 | 14 |
| 1393639_at | myosin X (predicted) | −4.95 | 0.00009 | 0.00811 | −0.59 | 0.67 | −1.50 | 14 |
| 1393790_at | HRAS-like suppressor | 5.44 | 0.00003 | 0.00432 | 0.44 | 1.35 | 1.35 | 14 |
| | (predicted) | | | | | | | |
| 1393798_at | alpha thalassemia/mental | −5.00 | 0.00008 | 0.00757 | −0.84 | 0.56 | −1.79 | 14 |
| | retardation syndrome X-linked | | | | | | | |
| | homolog (human) | | | | | | | |
| 1393804_at | similar to hypothetical protein | −6.79 | 0.00000 | 0.00073 | −0.85 | 0.56 | −1.80 | 14 |
| | FLJ22490 (predicted) | | | | | | | |
| 1393809_at | Tnf receptor-associated factor 6 | −8.48 | 0.00000 | 0.00009 | −0.90 | 0.53 | −1.87 | 14 |
| | (predicted) | | | | | | | |
| 1393811_at | similar to putative repair and | −6.08 | 0.00001 | 0.00178 | −0.79 | 0.58 | −1.73 | 14 |
| | recombination helicase | | | | | | | |
| | RAD26L | | | | | | | |
| 1393910_at | similar to Fam13a1 protein | −4.85 | 0.00011 | 0.00921 | −0.81 | 0.57 | −1.75 | 14 |
| | (predicted) | | | | | | | |
| 1393981_at | similar to KIAA0423 | −5.24 | 0.00005 | 0.00556 | −0.57 | 0.68 | −1.48 | 14 |
| | (predicted) | | | | | | | |
| 1394003_at | similar to DNA polymerase | −5.59 | 0.00002 | 0.00349 | −0.59 | 0.67 | −1.50 | 14 |
| | epsilon p17 subunit (DNA | | | | | | | |
| | polymerase epsilon subunit 3) | | | | | | | |
| | (Chromatin accessibility | | | | | | | |
| | complex 17) (HuCHRAC17) | | | | | | | |
| | (CHRAC-17) | | | | | | | |
| 1394220_at | Similar to hypothetical protein | 5.46 | 0.00003 | 0.00417 | 0.43 | 1.34 | 1.34 | 14 |
| | (predicted) | | | | | | | |
| 1394243_at | similar to spermine synthase | −6.11 | 0.00001 | 0.00175 | −0.60 | 0.66 | −1.51 | 14 |
| 1394436_at | sperm associated antigen 9 | −6.60 | 0.00000 | 0.00090 | −0.91 | 0.53 | −1.88 | 14 |
| | (predicted) | | | | | | | |
| 1394497_at | similar to TCF7L2 protein | −8.03 | 0.00000 | 0.00016 | −1.06 | 0.48 | −2.08 | 14 |
| 1394594_at | Transcribed locus | 5.09 | 0.00006 | 0.00671 | 0.42 | 1.34 | 1.34 | 14 |
| 1394715_at | Dicer1, Dcr-1 homolog | 5.14 | 0.00006 | 0.00627 | 0.54 | 1.46 | 1.46 | 14 |
| | (Drosophila) (predicted) | | | | | | | |
| 1394740_at | | 5.41 | 0.00003 | 0.00440 | 0.52 | 1.43 | 1.43 | 14 |
| 1394742_at | Transcribed locus | −5.73 | 0.00002 | 0.00289 | −0.98 | 0.51 | −1.98 | 14 |
| 1394746_at | hect (homologous to the E6-AP | −7.32 | 0.00000 | 0.00039 | −0.94 | 0.52 | −1.91 | 14 |
| | (UBE3A) carboxyl terminus) | | | | | | | |
| | domain and RCC1 (CHC1)-like | | | | | | | |
| | domain (RLD) 1 (predicted) | | | | | | | |
| 1394814_at | translocated promoter region | −6.13 | 0.00001 | 0.00171 | −0.63 | 0.64 | −1.55 | 14 |
| | (predicted) | | | | | | | |
| 1394849_at | Transcribed locus | −5.22 | 0.00005 | 0.00569 | −1.61 | 0.33 | −3.05 | 14 |
| 1394865_at | Transmembrane protein 7 | −7.85 | 0.00000 | 0.00019 | −0.92 | 0.53 | −1.90 | 14 |
| | (predicted) | | | | | | | |
| 1394965_at | enthoprotin | 5.30 | 0.00004 | 0.00503 | 0.40 | 1.32 | 1.32 | 14 |
| 1394969_at | Transcribed locus | 5.40 | 0.00003 | 0.00441 | 0.39 | 1.31 | 1.31 | 14 |
| 1394985_at | early endosome antigen 1 | −7.60 | 0.00000 | 0.00026 | −1.00 | 0.50 | −2.00 | 14 |
| | (predicted) | | | | | | | |
| 1395211_s_at | supervillin (predicted) | −8.74 | 0.00000 | 0.00007 | −0.98 | 0.51 | −1.97 | 14 |
| 1395237_at | eukaryotic translation initiation | −8.31 | 0.00000 | 0.00012 | −0.87 | 0.55 | −1.83 | 14 |
| | factor 5B | | | | | | | |
| 1395264_at | similar to Rap1-interacting | −6.85 | 0.00000 | 0.00067 | −0.95 | 0.52 | −1.93 | 14 |
| | factor 1 | | | | | | | |
| 1395331_at | similar to hypothetical protein | 4.84 | 0.00011 | 0.00945 | 0.31 | 1.24 | 1.24 | 14 |
| | CL25084 (predicted) | | | | | | | |
| 1395338_at | leucine-rich PPR-motif | 5.24 | 0.00005 | 0.00555 | 0.75 | 1.68 | 1.68 | 14 |
| | containing (predicted) | | | | | | | |
| 1395516_at | similar to hypothetical protein | −4.89 | 0.00010 | 0.00883 | −0.59 | 0.66 | −1.51 | 14 |
| | FLJ10154 (predicted) | | | | | | | |
| 1395565_at | COP9 signalosome subunit 4 | 5.55 | 0.00002 | 0.00376 | 0.40 | 1.32 | 1.32 | 14 |
| 1395610_at | similar to Hypothetical protein | 5.66 | 0.00002 | 0.00325 | 0.33 | 1.26 | 1.26 | 14 |
| | MGC30714 | | | | | | | |
| 1395616_at | similar to Ab2-008 (predicted) | −5.03 | 0.00007 | 0.00729 | −0.50 | 0.71 | −1.42 | 14 |
| 1395625_at | Transcribed locus | −6.03 | 0.00001 | 0.00187 | −0.76 | 0.59 | −1.70 | 14 |
| 1395739_at | similar to RIKEN cDNA | 5.05 | 0.00007 | 0.00698 | 0.54 | 1.46 | 1.46 | 14 |
| | C920006C10 (predicted) | | | | | | | |
| 1395814_at | Transcribed locus | −5.09 | 0.00006 | 0.00663 | −0.78 | 0.58 | −1.71 | 14 |
| 1395976_at | similar to phosphoinositol 4- | −6.37 | 0.00000 | 0.00126 | −0.57 | 0.67 | −1.49 | 14 |
| | phosphate adaptor protein-2 | | | | | | | |
| 1395981_at | helicase, ATP binding 1 | −5.76 | 0.00001 | 0.00276 | −0.62 | 0.65 | −1.54 | 14 |
| | (predicted) | | | | | | | |
| 1396036_at | Ral GEF with PH domain and | −6.67 | 0.00000 | 0.00084 | −1.04 | 0.49 | −2.06 | 14 |
| | SH3 binding motif 2 | | | | | | | |
| | (predicted) | | | | | | | |
| 1396063_at | DEK oncogene (DNA binding) | −4.82 | 0.00012 | 0.00952 | −0.63 | 0.65 | −1.55 | 14 |
| 1396100_at | similar to RIKEN cDNA | −5.15 | 0.00006 | 0.00610 | −0.56 | 0.68 | −1.47 | 14 |
| | 2010009L17 (predicted) | | | | | | | |
| 1396170_at | WW domain binding protein 4 | −7.78 | 0.00000 | 0.00020 | −0.77 | 0.59 | −1.71 | 14 |
| 1396187_at | Hypothetical protein | 5.14 | 0.00006 | 0.00622 | 0.51 | 1.43 | 1.43 | 14 |
| | LOC606294 | | | | | | | |
| 1396202_at | Transcribed locus | 4.97 | 0.00008 | 0.00795 | 0.52 | 1.44 | 1.44 | 14 |
| 1396403_at | | −9.07 | 0.00000 | 0.00005 | −1.01 | 0.50 | −2.02 | 14 |
| 1396803_at | similar to THO complex 2 | −7.09 | 0.00000 | 0.00050 | −0.90 | 0.54 | −1.86 | 14 |
| 1397203_at | PRP4 pre-mRNA processing | −6.18 | 0.00001 | 0.00162 | −0.67 | 0.63 | −1.59 | 14 |
| | factor 4 homolog B (yeast) | | | | | | | |
| | (predicted) | | | | | | | |
| 1397234_at | G patch domain containing 1 | −5.65 | 0.00002 | 0.00326 | −0.49 | 0.71 | −1.40 | 14 |
| | (predicted) | | | | | | | |
| 1397367_at | A disintegrin and | 5.05 | 0.00007 | 0.00698 | 0.47 | 1.38 | 1.38 | 14 |
| | metalloprotease domain 23 | | | | | | | |
| | (predicted) | | | | | | | |
| 1397508_at | similar to RIKEN cDNA | −5.08 | 0.00006 | 0.00671 | −0.62 | 0.65 | −1.54 | 14 |
| | 2310005B10 | | | | | | | |
| 1397552_at | echinoderm microtubule | −8.47 | 0.00000 | 0.00009 | −1.39 | 0.38 | −2.62 | 14 |
| | associated protein like 4 | | | | | | | |
| | (predicted) | | | | | | | |
| 1397627_at | diaphanous homolog 1 | −5.07 | 0.00007 | 0.00680 | −0.52 | 0.70 | −1.43 | 14 |
| | (Drosophila) (predicted) | | | | | | | |
| 1397647_at | solute carrier family 25 | 5.51 | 0.00003 | 0.00395 | 0.62 | 1.54 | 1.54 | 14 |
| | (mitochondrial carrier; | | | | | | | |
| | ornithine transporter) member | | | | | | | |
| | 15 (predicted) | | | | | | | |
| 1397669_at | Chemokine (C-C motif) | 5.78 | 0.00001 | 0.00271 | 0.51 | 1.43 | 1.43 | 14 |
| | receptor 6 (predicted) | | | | | | | |
| 1397674_at | eukaryotic translation initiation | −6.44 | 0.00000 | 0.00116 | −0.76 | 0.59 | −1.69 | 14 |
| | factor 3, subunit 8, 110 kDa | | | | | | | |
| | (predicted) | | | | | | | |
| 1397676_at | Similar to osteoclast inhibitory | −6.68 | 0.00000 | 0.00084 | −1.34 | 0.39 | −2.54 | 14 |
| | lectin | | | | | | | |
| 1397758_at | Similar to choline | −4.83 | 0.00011 | 0.00946 | −0.38 | 0.77 | −1.30 | 14 |
| | phosphotransferase 1; | | | | | | | |
| | cholinephosphotransferase 1 | | | | | | | |
| | alpha; | | | | | | | |
| | cholinephosphotransferase 1 | | | | | | | |
| 1397959_at | similar to RIKEN cDNA | −6.39 | 0.00000 | 0.00123 | −1.14 | 0.45 | −2.20 | 14 |
| | D130059P03 gene (predicted) | | | | | | | |
| 1398311_a_at | kinase D-interacting substance | 5.14 | 0.00006 | 0.00627 | 0.44 | 1.36 | 1.36 | 14 |
| | 220 | | | | | | | |
| 1398351_at | Ubiquitin specific protease 7 | −5.60 | 0.00002 | 0.00349 | −0.42 | 0.75 | −1.34 | 14 |
| | (herpes virus-associated) | | | | | | | |
| | (predicted) | | | | | | | |
| 1398420_at | Similar to E3 ubiquitin ligase | −5.33 | 0.00004 | 0.00483 | −0.94 | 0.52 | −1.92 | 14 |
| | SMURF2 (predicted) | | | | | | | |
| 1398436_at | ubiquitin specific protease 42 | −6.36 | 0.00000 | 0.00126 | −0.76 | 0.59 | −1.69 | 14 |
| | (predicted) | | | | | | | |
| 1398486_at | CDNA clone MGC: 93990 | −8.09 | 0.00000 | 0.00016 | −1.53 | 0.35 | −2.89 | 14 |
| | IMAGE: 7115381 | | | | | | | |
| 1398522_at | similar to Ab2-034 (predicted) | −4.92 | 0.00009 | 0.00832 | −0.51 | 0.70 | −1.42 | 14 |
| 1398553_at | similar to CGI-100-like protein | −6.91 | 0.00000 | 0.00062 | −1.68 | 0.31 | −3.20 | 14 |
| 1398834_at | mitogen activated protein | −4.94 | 0.00009 | 0.00828 | −0.32 | 0.80 | −1.25 | 14 |
| | kinase kinase 2 | | | | | | | |
| 1398926_at | prefoldin 1 (predicted) | −5.95 | 0.00001 | 0.00208 | −0.48 | 0.72 | −1.40 | 14 |
| 1398963_at | TAF10 RNA polymerase II, | −5.42 | 0.00003 | 0.00436 | −0.41 | 0.75 | −1.33 | 14 |
| | TATA box binding protein | | | | | | | |
| | (TBP)-associated factor | | | | | | | |
| | (predicted) | | | | | | | |
| 1399099_at | heterogeneous nuclear | −4.94 | 0.00009 | 0.00829 | −0.54 | 0.69 | −1.46 | 14 |
| | ribonucleoprotein U-like 1 | | | | | | | |
| | (predicted) | | | | | | | |
| 1399140_at | Transcribed locus | −5.16 | 0.00005 | 0.00597 | −0.49 | 0.71 | −1.40 | 14 |
| AFFX-BioB- | Biotin synthase | −4.89 | 0.00010 | 0.00879 | −0.64 | 0.64 | −1.56 | 14 |
| M_at | biotin synthesis, sulfur | | | | | | | |
| | insertion? | | | | | | | |
| AFFX- | dethiobiotin synthetase | −4.92 | 0.00009 | 0.00834 | −0.70 | 0.62 | −1.62 | 14 |
| BioDn-5_at | | | | | | | | |
| AFFX-r2-Ec- | dethiobiotin synthetase | −5.41 | 0.00003 | 0.00440 | −0.51 | 0.70 | −1.43 | 14 |
| bioD-5_at | | | | | | | | |

SLR: Estimated signal log-ratio (<0: down regulated gene, >0: up regulated gene).
Fold change: Estimated fold change corresponding to the parameter (<1: down regulated gene, >1: up regulated gene).
Affy fold change: Estimated fold change using the Affymetrix definition (<−1: down regulated gene, >1: up regulated gene)
df: Degrees of freedom (= number of arrays − number of estimated parameters)

### Example 2

18 healthy asthenozoospermic males (sperm motility <50%) are to be recruited in a fertility experiment. Other inclusion criteria are to be age (25-50), lack of ejaculation 2-5 days before sampling as well as a signed consent. Exclusion criteria are to be consumption of omega-3 products, use of carnitine, use of CoQ10, alcohol abuse and moderately severe co-morbid disease. The following variables are to be investigated at baseline and after 12 weeks: sperm motility, sperm count, sperm concentration, sperm morphology, sperm phospholipid fatty acid profile and pH. 6 males are to administer a marine phospholipid consisting of 700 mg/g EPA/DHA (ratio 2:1) daily for 12 weeks. 6 males are to administer olive oil and 6 males are to administer fish oil as described in the prior art [42] for 12 weeks. After 12 weeks administering marine phospholipids an improvement in sperm motility, sperm count, sperm concentration, sperm morphology is to be found compared to both placebo and fish oil. An increase in DHA level in the sperm phospholipid is to be found compared to placebo and fish oil as well.

### Example 3

Sprague-Dawley rats were fed different omega-3 fatty acid composition (TG, PL 1 and PL 2) as well as placebo (control) for 30 days. The rat feed was prepared using AIN-93 except that soybean oil was removed from the feed. The pelleted AIN-93 diet was ground and the marine lipid compositions (PL 1 and PL 2) as well as fish oil (TG oil) and control were added to this ground feed. The marine lipid compositions were prepared using enzymatic (lipase) catalyzed transesterification of soy lecithin with fish oil fatty acids according to the method described in Example 1. The concentration of EPA, DHA and 18:3 n-3 in the different diets can be seen in the table below (table 3).

TABLE 3
Amount of different fatty acid in the final feed products

| | | g/100 g | g/100 g | g/100 g | SUM g/100 g |
|---|---|---|---|---|---|
| | | EPA | DHA | 18:3n3 | EPA + DHA + 18:3n3 |
| Control | T4 | 0 | 0 | 0.26 | 0.26 |
| TG Oil | T1 | 0.61 | 0.39 | 0.24 | 1.23 |
| PL 1 | T2 | 0.61 | 0.35 | 0.26 | 1.22 |
| PL 2 | T3 | 0.24 | 0.73 | 0.26 | 1.23 |

Thirty six newly weaned male Sprague Dawley rats (start weight 168±11 g) were used in the experiment. The rats were initially given low-essential oil rat feed, containing 20 g of sunflower oil and 10 g of flaxseed oil per kg of feed, for one week. After the first week, modified AIN-93 diet powder without the test oil was given to rats ad libitum until the start of the experiment. Feeding of rats was stopped 12 hours before the sampling, 30 days after the start of feeding. Each rat was individually anaesthetized with carbon dioxide, weighed and euthanized with cervical dislocation. The brain, liver, heart, plasma, testis and adipose tissue were removed from the rats and analyzed for fatty acid composition in the total lipids, the phospholipids as well as the sn-2 position of the phospholipids. Table 4, 5 and 6 shows the fatty acid analysis of the testis total lipids, phospholipids and phospholipids sn-2 position, respectively.

TABLE 4
Fatty acid profile of total lipids in testis

| nmoles | | | | | | | | | | | | | |
|---|---|---|---|---|---|---|---|---|---|---|---|---|---|
| FA/mg | | | n3 | n6 | | | | | | | 20:3 | | |
| lipids | 18:4 | 20:5 | 18:3 | 18:3 | 22:6 | 16:1 | 20:4 | 18:2 | 22:5 | 20:3 | n9 | 22:4 | 18:1 |
| TG oil | 3.2 | 17.0 | 11.7 | 1.8 | 56.6 | 47.6 | 280.1 | 195.1 | 6.9 | 285.6 | 3.7 | 56.2 | 326.8 |
| PL- | 0.6 | 10.8 | 9.2 | 1.7 | 52.4 | 35.6 | 294.9 | 210.1 | 6.1 | 294.2 | 3.4 | 61.3 | 348.3 |
| EPA | | | | | | | | | | | | | |
| PL- | 2.4 | 6.1 | 1.7 | 2.0 | 56.0 | 0.0 | 310.8 | 156.5 | 2.4 | 345.3 | 6.2 | 59.5 | 332.5 |
| DHA | | | | | | | | | | | | | |
| control | 1.8 | 1.1 | 1.6 | 2.0 | 27.2 | 0.0 | 335.5 | 162.2 | 1.3 | 319.1 | 5.8 | 78.7 | 330.1 |

TABLE 5
Fatty acid profile of the phospholipids from the testis

| nmoles FA/mg | | | | | | | | |
|---|---|---|---|---|---|---|---|---|
| lipids | 20:5 | 22:6 | 20:4 | 18:2 | 22:5 n3 | 22:5 n6 | 22:4 | 18:1 |
| TG oil | 3.18 | 23.00 | 173.10 | 55.30 | 1.55 | 234.24 | 20.50 | 118.11 |
| PL-EPA | 3.71 | 31.41 | 227.44 | 82.56 | 2.05 | 317.90 | 28.27 | 143.41 |
| PL-DHA | 3.339 | 46.79 | 335.87 | 127.50 | 2.04 | 265.36 | 45.48 | 155.71 |
| control | 0.430 | 14.08 | 204.27 | 49.39 | 0.36 | 161.20 | 35.70 | 118.18 |

TABLE 6
Fatty acid profile of the testis in the phospholipids sn-2 position.

| sn-2 | 20:5 | 22:6 | 20:4 | 18:2 | 22:5 | 22:4 | 18:1 |
|---|---|---|---|---|---|---|---|
| TAG | 2.995 | 21.886 | 167.602 | 60.785 | 224.444 | 20.505 | 109.306 |
| EPA | 3.430 | 28.412 | 210.792 | 77.228 | 290.642 | 28.274 | 120.382 |
| DHA | 2.582 | 41.600 | 304.767 | 110.468 | 244.121 | 45.479 | 137.898 |
| CTRL | 0.418 | 13.662 | 202.159 | 50.934 | 157.274 | 35.695 | 113.768 |

### Example 4

The effect of dietary supplementation of omega-3 phospholipids on the prevention of obesity is to be investigated. 5 rats will be fed a control+high fat diet containing essentially no EPA/DHA for 2 weeks, 5 rats will be fed a marine phospholipid high fat diet and 5 rats will be fed a fish oil high fat diet also for 2 weeks. It is to be found that the weight gain and/or accumulation of adipose tissue are significantly larger in the control high fat diet, compared to other two diets. It is to be found that the weight gain and/or accumulation of adipose tissue is significantly larger in the fish oil high fat diet compared to the marine phospholipid high fat diet.

### Example 5

The effect of dietary supplementation of omega-3 phospholipids (700 mg omega-3/day, EPA:DHA=2:1) on delayed onset muscle soreness (DOMS) in human subjects is to be investigated. 10 subjects will receive omega-3 phospholipids and 10 subjects will receive fish oil for 30 days prior to exercise. Next, DOMS is to be induced e.g. by 50 maximal isokinetic eccentric elbow flexion contractions. DOMS is to be measured by asking the subjects about pain, swelling and muscle strength as well as measuring typical markers for DOMS and muscle damage such as creatine kinase. It is to be observed that the use of omega-3 phospholipids significantly reduces DOMS compared to fish oil.

### Example 6

The immediate effect of administration of omega-3 phospholipids in humans with sustained ventricular tachycarida is to be investigated. 10 patients with implanted cardioverter defibrillators and repeated episodes of documented, sustained ventricular tachycardia are to be enrolled in a study. Omega-3 fatty acids 3.8 g are to be infused either in the form of fish oil (N=5) or marine phospholipids (N=5). Sustained ventricular tachycardia is to be induced using paced cycle lengths of variable length in the patients in both groups. It is to be found that the group receiving omega-3 phospholipids have fewer cases of ventricular tachycardia than the group receiving omega-3 fish oil.

### Example 6

The heart from the rats tested in example 3 were isolated and analyzed for fatty acid profile in the total lipids, phospholipids and the sn-2 position on the phospholipids (table 7-9). The results show an increase of omega-3 fatty acids in the phospholipids isolated from the heart.

TABLE 7
Fatty acid composition on total lipids in heart.

| nmoles | | | | | | | | | | | | | |
|---|---|---|---|---|---|---|---|---|---|---|---|---|---|
| FA/mg | | | n3 | n6 | | | | | | | 20:3 | | |
| lipids | 18:4 | 20:5 | 18:3 | 18:3 | 22:6 | 16:1 | 20:4 | 18:2 | 22:5 | 20:3 | n9 | 22:4 | 18:1 |
| TG oil | 2.0 | 44.7 | 20.2 | 0.9 | 314.0 | 49.7 | 279.6 | 787.9 | 4.0 | 6.8 | | 3.0 | 288.9 |
| PL- | 1.5 | 45.0 | 25.6 | 0.8 | 314.4 | 44.6 | 300.4 | 789.0 | 4.4 | 7.2 | | 3.3 | 276.6 |
| EPA | | | | | | | | | | | | | |
| PL- | 0.2 | 29.1 | 9.9 | 0.4 | 372.1 | 30.1 | 291.0 | 690.3 | 9.0 | 6.3 | 1.5 | 4.4 | 237.0 |
| DHA | | | | | | | | | | | | | |
| control | 0.0 | 2.9 | 7.2 | 0.7 | 209.9 | | 495.8 | 756.2 | 14.7 | 6.2 | 2.4 | 30.3 | 289.5 |

TABLE 8
Fatty acid composition of phospholipids in heart

| nmoles FA/mg | | n3 | | | | | | 20:3 | | |
|---|---|---|---|---|---|---|---|---|---|---|
| lipids | 20:5 | 18:3 | 22:6 | 20:4 | 18:2 | 22:5 | 20:3 | n9 | 22:4 | 18:1 |
| TG oil | 35.0 | 6.4 | 286.3 | 240.3 | 877.5 | 4.2 | 5.1 | | 3.2 | 119.6 |
| PL-EPA | 27.5 | 6.0 | 261.6 | 205.1 | 754.6 | 3.4 | 4.2 | | 2.6 | 96.3 |
| PL-DHA | 20.8 | 4.1 | 331.1 | 227.6 | 570.3 | 7.9 | 5.0 | 1.2 | 4.1 | 115.7 |
| control | 1.6 | 3.1 | 187.1 | 348.8 | 505.9 | 13.7 | 3.2 | 1.9 | 22.0 | 128.6 |

TABLE 9
Fatty acid composition in the SN-2 position in the heart.

| sn-2 | 20:5 | n3 18:3 | 22:6 | 20:4 | 18:2 | 22:5 | 20:3 | 20:3 n9 | 22:4 | 18:1 |
|---|---|---|---|---|---|---|---|---|---|---|
| TAG | 28.6 | 5.3 | 215.4 | 164.9 | 741.7 | 2.9 | 3.2 | | 3.2 | 68.2 |
| EPA | 22.2 | 4.3 | 193.4 | 131.5 | 601.8 | 2.1 | 2.2 | | 2.5 | 52.5 |
| DHA | 17.6 | 3.4 | 256.7 | 160.4 | 519.5 | 5.7 | 3.2 | 1.2 | 4.1 | 70.0 |
| CTRL | 1.4 | 1.9 | 163.7 | 283.9 | 467.0 | 8.4 | 2.9 | 1.9 | 21.9 | 96.0 |

### Example 8

The effect of dietary supplementation of omega-3 phospholipids on physical endurance is to be investigated in a experiment with rats (N=6). Rats will be fed one of two diets containing 20 energy % of fat for 5 to 10 weeks. Control diet containing essentially no EPA/DHA will be supplemented with 10 energy % of olive oil and the test diet will be supplemented with 10 energy % of marine phospholipid. It is to be found that rats consuming the marine phospholipid diet will demonstrate increased submaximal endurance in a treadmill running test.

### Example 9

The effect of dietary supplementation of omega-3 phospholipids on inflammation is to be investigated in a experiment with rats (n=6-12). The experiment will use a control diet containing essentially no EPA/DHA, a positive control diet containing fish oil and a test diet containing marine phospholipids (EPA:DHA; 2:1). The effect of marine phospholipids on the inflammatory process will be examined first by analyzing the fatty acid profile of inflammatory cell membrane phospholipids (e.g. monocytes and macrophages). The effect of omega-3 fatty acids on inflammation is mediated through the link between phospholipid stores of inflammatory cells [43]. Inflammatory cells typically contain a high proportion of arachidonic acid (AA; C20:4 omega-6) and low proportions of omega-3 fatty acids. Dietary supplementation with omega-3 fatty acids decreases the omega-6:omega-3 ratio of the inflammatory cells thus decreasing the inflammatory potential of the inflammatory cells. This is a desirable change in humans with chronic inflammatory diseases such as rheumatoid arthritis, inflammatory bowel disease and atopic diseases such as asthma. It is to be observed that marine phospholipids reduce omega-6:omega-3 ratio in blood inflammatory cells such as in monocytes significantly compared to the control and fish oil supplemented diets. In the second stage of the experiment monocytes harvested from the experimental animals will be challenged with lipopolysaccharide (LPS), bacterial cell surface antigen that triggers and inflammatory response in monocytes, and the pro-inflammatory cytokine response (TNFα, IL-1β, IL-6 and IL-8) will be measured. It is to be observed that marine phospholipids are more effective in reducing inflammatory cytokine production in monocytes than the control and fish oil containing diet.

### Examples 10

The purpose of the study is to differentiate the effect of omega-3 phospholipids, omega-3 triglycerides and control on inflammation, blood lipids, insulin resistance and oxidative stress. Different forms of omega-3 fatty acids are given to Zücker diabetic fatty rats (ZDF rats), an animal model relevant to human obesity, for 5 weeks. The omega-3 rich phospholipids were prepared according to the method in example 12. The data is presented in Table 10. It is observed that there are no difference between the treatments on insulin levels and HOMA estimates. However, it is observed that in the phospholipid group are the most efficient formulation in improving plasma glucose levels. Elevated plasma glucose levels are one of the signs/symptoms of metabolic syndrome. Further, it is expected that omega-3 phospholipids are the most efficient formulation in improving blood lipids such as HDL, LDL, triglycerides and free fatty acids, for reducing inflammatory markers such as TNF alfa, IL-1 beta, IL-6, IL-10, TGF beta and fibrinogen in plasma, and for reducing markers of oxidative stress such as PUFA hydroperoxides and 15-F2t-Isoprostanes in plasma and tissues (subcutaneous and visceral adipose tissue, liver, brain and heart).

TABLE 10
The effect of control, omega-3 phospholipids and omega-3 triglycerides
on markers of insulin resistance in ZDF rats.

| | Glycemia | Glycemia | Insulin | HOMA |
|---|---|---|---|---|
| | mg/dl | mmoles/L | micronU/ml | IR |
| contr | 346.667 | 19.259 | 3.767 | 3.206 |
| | 48.578 | 2.699 | 1.787 | 1.658 |
| TAG | 309.833 | 17.213 | 1.667 | 1.273 |
| | 7.195 | 0.400 | 0.333 | 0.247 |
| PL | 294.667 | 16.370 | 1.777 | 1.318 |
| | 29.864 | 1.659 | 0.478 | 0.467 |

### Example 11

The effect of omega-3 rich phospholipids on collagen induced rheumatoid arthritis is to be investigated in a therapeutic animal model. The omega-3 rich phospholipids were prepared according to the method in example 12. Rheumatoid arthritis (RA) is considered to be a chronic, inflammatory autoimmune disorder that causes the immune system to attack the joints. It is expected that omega-3 phospholipids are more efficient in increasing clinical arthritis scores than omega-3 triglycerides and placebo.

### Example 12

50 g of soy lecithin from American Lecithin Company Inc (Oxford, Conn., USA), 40 g of TL-IM lipase from Novozymes (Bagsvaerd, Denmark) and 5 g of water (adjusted to pH=8 using NaOH) were mixed in a reaction vessel at 50° C. for 24 hours. Next, 10 g of free fatty acids containing 10% EPA and 50% DHA from Napro Pharma (Brattvaag, Norway) was added, followed by application of vacuum to the reaction vessel. After 72 hours the reaction was terminated and the phospholipid mixture was analyzed using HPLC and GC. The results showed that the relationship between PC/LPC/GPC was 65/35/0, and that the content of EPA and DHA was around 10% and 12%, respectively. Next, 20 g of sardine oil was added to the reaction mixture which comprised of 18% EPA and 12% DHA (relative GC peak area), followed by molecular distillation. The final product contained around 70% acetone insolubles, around 30% triglycerides and traces of free fatty acids.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

### REFERENCES

[1] Doig S and Diks R M M. Eur J Lipid Sci Technol 105 (2003) 359.
[2] Adlercreutz P, Lyberg A M and Adlercreutz D. Eu. J Lipid. Sci Technol 105 (2003) 638.
[3] Peng L, Xu X, Mu H, Høy C E and Adler-Nissen J. Enzyme Microb Technol 31 (2002) 532.
[4] Haraldsson G G and Thorarensen A, JAOCS 75 (1999) 1143.
[5] Guo Z, Vikbjerg A F and Xu X. Biotech Adv. 23 (2005) 203.
[6] US 20040234587.
[7] U.S. Pat. No. 6,036,992.
[8] U.S. Pat. No. 5,434,183.
[9] U.S. Pat. No. 5,853,747.
[10] US 20050130937.
[11] Lemaitre-Delaunay D, Pachiaudi C, Laville M, Pousin J, Armstrong M and Lagarde M. J. Lipid Res. 40 (1999) 1867.
[12] Wijendran V, Huang M, Diau G, Boehm G, Nathanielsz P W and Brenna J T. Pediatr. Res. 51 (2002) 365.
[13] Calder P. Nutr. Res. 24 (2004) 361.
[14] Balk E M, Lichtenstein A H, Chung M, Kupelnick B, Chew P, Lau J. Atherosclerosis 9 (2006)
[15] Wainwright, P. Br J Nutr. 83 (2000) 337.
[16] Concuer J A, Martin J B, Tummon I, Watson L and Tekpetey F. Lipids 35 (2000) 149.
[17] Youdim K A, Martin A and Joseph J A. Int J Dev Neurosci 18 (2000) 383.
[18] Nissen H P and Kreysel H W. Andrologia 15 (1983) 264
[19] Brinsko S, Varner D D, Love C C, Blanchard T L, Day B C and Wilson M E. Theriogeneology 63 (2005) 1519.
[20] Alexander J W. Nutrition 14 (1998) 627.
[21] Belluzi A, Boschi S, Brignola C, Munarini A, Cariani G and Miglio F. Am J Clin Nutr 71 (2000) 339.
[22] Kremer J M. Am J Clin Nutr 71 (2000) 249.
[23] Aggarwal B B, Sethi G, Nair A and Ichikawa H. Current Signal Transduction Therapy 1 (2006) 25-52.
[24] Zhao Y, Joshi-Barve J., Barve S., Chen L. J. Am. Coll. Nutr 23 (2004) 71-78.
[25] Calder P. J. Nutr. Res. 24 (2004) 761.
[26] Kramer J A, LeDeaux J, Butteiger D, Young T, Crankshaw C, Harlow H, Kier L and Bhat G. J. Nutr, 133 (2003) 57.
[27] Ruziskova J, Rossmeisl M, Prazak T, Flachs P, Sponarova J, Vecka M, Tvrzicka E, Bryhn M and Kopecky J. Lipids 39 (2004) 1177.
[28] Mandl, J. P. Journal of Reproduction and Fertility (1972), 31(2), 263-9.
[29] Bannach F G, Gutierrez-Fernandez A, Parmer R J and Miles L A. J Thromb Haemost. 2 (2004) 2205.
[30] Choi Y. Adv Exp Med Biol. 560 (2005) 77.
[31] Leon K G and Karsan A. Histol Histopathol. 15 (2000) 1303.
[32] Armstrong R B. Med. Sci. Sports Exerc 22 (1990) 529.
[33] Smith L L. Med. Sci. Sports Exersc 25 (1991) 542.
[34] Andersson A, Nalsen C, Tengblad A, Vessby B. Am J Clin Nutr 76 (2002) 1222.
[35] Bruckner G, Webb P, Greeenwell L Chow C, Richardson D, Atherosclerosis 66 (1987) 237.
[36] Ohyanagi M and Iwasaki T. Mol Cell Biochem. 160-161 (1996) 153.
[37] Rebouche C J. Am. J Clin Nutr. 54 (1991) 1147S.
[38] Reda E, D'Iddio S, Nicolai R, Benatti P, Calvani M. Acta Diabetol. 40 (2003) S106.
[39] Kris-Ethertom P M, Harris W S, Appel L J, Circulation 106 (2002) 2747.
[40] Screpf R, Limmert T, Weber P C, Theisen K, Sellmayer A. Lancet 363 (2004) 1141.
[41] Sauer L A, Dauchy R T, Blask D E, Krause J A, Davidson L K, Dauchy E M. Journal of Nutrition 135 (2005) 2124
[42] WO2004056370
[43] Calder, P C. Lipids 38 (2003) 343.

1. A method of treating a subject by administration of a marine phospholipid composition comprising:
administering a marine phospholipid composition to said subject under conditions such that a desired condition is improved, wherein said conditions is selected from the group consisting of fertility, physical endurance, sports performance, muscle soreness, inflammation, auto-immune stimulation, metabolic syndrome, obesity and type II diabetes.

2. The method of claim 1, wherein said subject is a human.

3. The method of claim 1, wherein said subject is a companion animal.

4. The method of claim 1, wherein said marine phospholipid composition comprises phospholipids having the following structure: 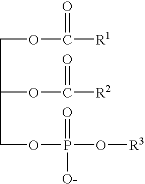
wherein R1 is OH or a fatty acid, R2 is OH or a fatty acid, and R3 is a mixture of H, choline, ethanolamine, inositol and serine, said phospholipid having at least 1% of omega-3 fatty acid moieties at positions R1 and/or R2.

5. The method of claim 4, wherein said phospholipid composition comprises from about 20-50% of OH at positions R1 and/or R2.

6. The method of claim 4, wherein said phospholipid composition is prepared from natural marine phospholipids isolated from a marine organism.

7. The method of claim 4, wherein said phospholipid composition is enzymatically prepared by reacting lecithin with DHA and EPA in the presence of an enzyme.

8. The method of claim 7, wherein said lecithin is soybean or egg lecithin.

9. The method of claim 4, wherein said omega-3 fatty acid moieties are selected from the group of EPA and DHA and combination thereof.

10. The method of claim 4, wherein said effective amount of said phospholipid composition comprises from about 300 to about 1000 mg omega-3 fatty acids.

11. The method of claim 4, wherein said phospholipid composition is administered orally.

12. The method of claim 4, wherein said phospholipid composition is provided in a gel capsule or pill.

13. The method of claim 4, wherein said phospholipid composition further comprises a triglyceride carrier.

14. The method of claim 1, wherein said male is a human.

15. A method of treating a subject by administration of a marine phospholipid composition comprising:
administering a marine phospholipid composition to a subject under conditions such that an undesirable condition is prevented, wherein said undesirable condition is selected from the group consisting of weight gain, infertility, obesity, metabolic syndrome, diabetes type II, mortality in subjects with a high risk of sudden cardiac death, and induction of sustained ventricular tachycardia.

16. The method of claim 15, wherein said subject is at risk for developing a condition selected from the group consisting of weight gain, obesity, metabolic syndrome, diabetes type II, mortality in subjects with a high risk of sudden cardiac death, and induction of sustained ventricular tachycardia.

17. The method of claim 15, wherein said subject is a human.

18. The method of claim 15, wherein said subject is a companion animal.

19. The method of claim 15, wherein said marine phospholipid composition comprises phospholipids having the following structure: 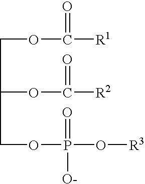
wherein R1 is OH or a fatty acid, R2 is OH or a fatty acid, and R3 is a mixture of H, choline, ethanolamine, inositol and serine, said phospholipid having at least 1% of omega-3 fatty acid moieties at positions R1 and/or R2.

20. The method of claim 19, wherein said phospholipid composition comprises from about 20-50% of OH at positions R1 and/or R2.

21. The method of claim 19, wherein said phospholipid composition is prepared from natural marine phospholipids isolated from a marine organism.

22. The method of claim 19, wherein said phospholipid composition is enzymatically prepared by reacting lecithin with DHA and EPA in the presence of an enzyme.

23. The method of claim 22, wherein said lecithin is soybean or egg lecithin.

24. The method of claim 15, wherein said omega-3 fatty acid moieties are selected from the group of EPA and DHA and combination thereof.

25. The method of claim 15, wherein said effective amount of said phospholipid composition comprises from about 300 to about 1000 mg omega-3 fatty acids.

26. The method of claim 15, wherein said phospholipid composition is administered orally.

27. The method of claim 15, wherein said phospholipid composition is provided in a gel capsule or pill.

28. The method of claim 15, wherein said phospholipid composition further comprises a triglyceride carrier.
